SEQUENCE LISTING (33 sequences)

SEQ ID NO: 1 - protein, 11 aa, Artificial Sequence
  CDR-L1
KASQGINNYLS

SEQ ID NO: 2 - protein, 7 aa, Artificial Sequence
  CDR-L2
RANRLVD

SEQ ID NO: 3 - protein, 7 aa, Artificial Sequence
  CDR-L2
RANRLVS

SEQ ID NO: 4 - protein, 9 aa, Artificial Sequence
  CDR-L3
LQYDEFPLT

SEQ ID NO: 5 - protein, 10 aa, Artificial Sequence
  CDR-H1
GYRFTDYVIN

SEQ ID NO: 6 - protein, 11 aa, Artificial Sequence
  CDR-H2
GQIYPGSDSFH

SEQ ID NO: 7 - protein, 11 aa, Artificial Sequence
  CDR-H2
GQIYPGSDTFH

SEQ ID NO: 8 - protein, 11 aa, Artificial Sequence
  CDR-H2
GQIYPGSDAFH

SEQ ID NO: 9 - protein, 7 aa, Artificial Sequence
  CDR-H3
FFEGLAY

SEQ ID NO: 10 - protein, 107 aa, Artificial Sequence
  Ab0001-HuV3-VL
DIQMTQSPSSLSASVGDRVTITCKASQGINNYLSWYQQKPGKAPKSLIYRANRLVDGVPSRFSGSGSGQDYTLTISSLQPEDFATYYCLQYDEFPLTFGGGTKVEIK

SEQ ID NO: 11 - protein, 107 aa, Artificial Sequence
  Ab0001-HuV4-VL
DIQMTQSPSSLSASVGDRVTITCKASQGINNYLSWYQQKPGKAPKSLIYRANRLVDGVPSRFSGSGSGTDYTLTISSLQPEDFATYYCLQYDEFPLTFGGGTKVEIK

SEQ ID NO: 12 - protein, 107 aa, Artificial Sequence
  Ab0001-HuV5-VL
DIQMTQSPSSLSASVGDRVTITCKASQGINNYLSWYQQKPGKAPKSLIYRANRLVDGVPSRFSGSGSGTDFTLTISSLQPEDFATYYCLQYDEFPLTFGGGTKVEIK

SEQ ID NO: 13 - protein, 107 aa, Artificial Sequence
  Ab0001-HuV8-VL
DIQMTQSPSSLSASVGDRVTITCKASQGINNYLSWYQQKPGKAPKSLIYRANRLVSGVPSRFSGSGSGTDFTLTISSLQPEDFATYYCLQYDEFPLTFGGGTKVEIK

SEQ ID NO: 14 - protein, 116 aa, Artificial Sequence
  Ab0001-HuV4-VH
QVQLVQSGAEVKKPGSSVKVSCKASGYRFTDYVINWVRQAPGQGLEWMGQIYPGSDSFHYNQKFQGRATLTADKSTNTAYMELSSLRSEDTAVYYCARFFEGLAYWGQGTLVTVSS

SEQ ID NO: 15 - protein, 116 aa, Artificial Sequence
  Ab0001-HuV5-VH
QVQLVQSGAEVKKPGSSVKVSCKASGYRFTDYVINWVRQAPGQGLEWMGQIYPGSDTFHYNQKFQGRATLTADKSTNTAYMELSSLRSEDTAVYYCARFFEGLAYWGQGTLVTVSS

SEQ ID NO: 16 - protein, 116 aa, Artificial Sequence
  Ab0001-HuV6-VH
QVQLVQSGAEVKKPGSSVKVSCKASGYRFTDYVINWVRQAPGQGLEWMGQIYPGSDAFHYNQKFQGRATLTADKSTNTAYMELSSLRSEDTAVYYCARFFEGLAYWGQGTLVTVSS

SEQ ID NO: 17 - protein, 222 aa, Artificial Sequence
  Ab0001-HuV3-LC
DIQMTQSPSSLSASVGDRVTITCKASQGINNYLSWYQQKPGKAPKSLIYRANRLVDGVPSRFSGSGSGQDYTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 18 - protein, 222 aa, Artificial Sequence
  Ab0001-HuV4-LC
DIQMTQSPSSLSASVGDRVTITCKASQGINNYLSWYQQKPGKAPKSLIYRANRLVDGVPSRFSGSGSGTDYTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 19 - protein, 222 aa, Artificial Sequence
  Ab0001-HuV5-LC
DIQMTQSPSSLSASVGDRVTITCKASQGINNYLSWYQQKPGKAPKSLIYRANRLVDGVPSRFSGSGSGTDFTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 20 - protein, 222 aa, Artificial Sequence
  Ab0001-HuV8-LC
DIQMTQSPSSLSASVGDRVTITCKASQGINNYLSWYQQKPGKAPKSLIYRANRLVSGVPSRFSGSGSGTDFTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 21 - protein, 446 aa, Artificial Sequence
  Ab0001-HuV4-HC
QVQLVQSGAEVKKPGSSVKVSCKASGYRFTDYVINWVRQAPGQGLEWMGQIYPGSDSFHYNQKFQGRATLTADKSTNTAYMELSSLRSEDTAVYYCARFFEGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 446 aa, Artificial Sequence
  Ab0001-HuV5-HC
QVQLVQSGAEVKKPGSSVKVSCKASGYRFTDYVINWVRQAPGQGLEWMGQIYPGSDTFHYNQKFQGRATLTADKSTNTAYMELSSLRSEDTAVYYCARFFEGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 446 aa, Artificial Sequence
  Ab0001-HuV6-HC
QVQLVQSGAEVKKPGSSVKVSCKASGYRFTDYVINWVRQAPGQGLEWMGQIYPGSDAFHYNQKFQGRATLTADKSTNTAYMELSSLRSEDTAVYYCARFFEGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 22 aa, Artificial Sequence
  N-terminal light chain signal peptide
MDMRVPAQLLGLLLLWLRGARC

SEQ ID NO: 25 - protein, 8 aa, Artificial Sequence
  Sortase recognition motif and spacer sequence
GALPETGG

SEQ ID NO: 26 - protein, 19 aa, Artificial Sequence
  N-terminal heavy chain signal peptide
MEFGLSWVFLVAILKGVQC

SEQ ID NO: 27 - protein, 214 aa, Artificial Sequence
  Ab0001-HuV3-LC
DIQMTQSPSSLSASVGDRVTITCKASQGINNYLSWYQQKPGKAPKSLIYRANRLVDGVPSRFSGSGSGQDYTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 214 aa, Artificial Sequence
  Ab0001-HuV4-LC
DIQMTQSPSSLSASVGDRVTITCKASQGINNYLSWYQQKPGKAPKSLIYRANRLVDGVPSRFSGSGSGTDYTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 214 aa, Artificial Sequence
  Ab0001-HuV5-LC
DIQMTQSPSSLSASVGDRVTITCKASQGINNYLSWYQQKPGKAPKSLIYRANRLVDGVPSRFSGSGSGTDFTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 214 aa, Artificial Sequence
  Ab0001-HuV8-LC
DIQMTQSPSSLSASVGDRVTITCKASQGINNYLSWYQQKPGKAPKSLIYRANRLVSGVPSRFSGSGSGTDFTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 222 aa, Artificial Sequence
  Ab0002-LC
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 32 - protein, 451 aa, Artificial Sequence
  Ab0002-HC
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 214 aa, Artificial Sequence
  Ab0002-LC
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0101266 A1
(43) Pub. Date: Mar. 30, 2023

(54) ANTI-TROP2 ANTIBODIES, ANTIBODY-DRUG CONJUGATES, AND APPLICATION OF THE SAME

(71) Applicant: GENEQUANTUM HEALTHCARE (SUZHOU) CO., LTD., Suzhou, Jiangsu (CN)

(72) Inventors: Gang QIN, Suzhou (CN); Dong YANG, Suzhou (CN); Lili SHI, Suzhou (CN)

(21) Appl. No.: 17/758,269

(22) PCT Filed: Dec. 31, 2020
(86) PCT No.: PCT/CN2020/142009

§ 371 Date: Jun. 30, 2022

(30) Foreign Application Priority Data
Dec. 31, 2019 (CN) PCT/CN2019/130780

(51) Int. Cl. C07K 16/30; A61K 47/68; A61P 35/00

(52) U.S. Cl. CPC C07K 16/30; A61K 47/6851; A61K 47/6849; A61K 47/6889; A61K 47/6817; A61P 35/00; A61K 2039/505

(57) ABSTRACT

Provided are novel anti-TROP2 antibodies, novel antibody-drug conjugates, and methods for preparing the same, as well as applications of the antibodies and antibody-drug conjugates for therapeutic purpose.

This application claims the priority to International Patent Application No. PCT/CN2019/130780, filed on Dec. 31, 2019, the disclosure of which is incorporated herein by reference in its entirety.

### TECHNICAL FIELD

The present disclosure relates to the biopharmaceutical field. In particular, provided are anti-TROP2 antibodies, antibody-drug conjugates, and methods for preparing the same, as well as applications of the antibodies and antibody-drug conjugates for therapeutic purpose.

### BACKGROUND

Trophoblast antigen 2 (TROP2) (also known as GA733-1, EGP-1, MIS1) is a single pass transmembrane glycoprotein, encoded by the gene TACSTD 2 (tumor-associated calcium signal transducer 2) (Bignotti E et al., BMC Clinical Pathology, 2012; 12: 22). It comprises an N-terminal leader peptide, an extracellular domain, a transmembrane domain, an intracellular domain and a cytoplasmic tail (Fornaro M et al., International Journal of Cancer, 1995, 62(5): 610-618). TROP2 transduces intracellular calcium signals, which in turn activate a variety of signaling pathways important for cell proliferation, survival, migration and invasion. TROP2 is highly expressed in a variety of late stage epithelial carcinomas, including pancreatic cancer, gastric cancer, and colorectal cancer. TROP2 overexpression is associated with poor prognosis of cancer patients as well as increased tumor aggressiveness and metastasis (Shvartsur A, Bonavida B. Genes & Cancer, 2015: 84-105). Therefore, TROP2 is a very attractive and potential therapeutic target for late stage carcinomas, such as epithelial carcinomas (Cubas R, et al., Biochimica et Biophysica Acta, 2009, 1796(2): 309-314). Anti-TROP2 antibodies hold great potential in the treatment of late stage carcinomas.

Several antibodies, antibody-drug conjugates (ADCs), and combination therapies against TROP2 have been reported. One of the ADCs in clinical trials for cancer therapy is Sacituzumab Govitecan (IMMU-132 or hRS7-SN-38, Immunomedics, U.S. Pat. Nos. 9,770,517 and 10,413,539), in which the humanized anti-TROP2 antibody hRS7 is conjugated to the toxic drug SN-38.

While TROP2 has potential as a therapeutic target, much is unknown regarding the preferred disease type, preferred therapeutic combinations, drug-delivery methods, and side effect profiles. There is a need for new alternative TROP2-targeted therapeutics.

### SUMMARY

In an aspect, provided is an anti-TROP2 antibody or antigen-binding fragment thereof, comprising a light chain variable region (V_{L}) and a heavy chain variable region (V_{H}), wherein the V_{L} comprises:
(i) CDR-L1 having an amino acid sequence of SEQ ID No. 1,
(ii) CDR-L2 having an amino acid sequence of SEQ ID No. 2 or SEQ ID No. 3,
(iii) CDR-L3 having an amino acid sequence of SEQ ID No. 4; and
wherein the V_{H} comprises:
(i) CDR-H1 having an amino acid sequence of SEQ ID No. 5,
(ii) CDR-H2 having an amino acid sequence of SEQ ID No. 6, SEQ ID No. 7 or SEQ ID No. 8, and
(iii) CDR-H3 having an amino acid sequence of SEQ ID No. 9.

In some embodiments, the V_{L} has an amino acid sequence having at least about 85%, at least about 90%, at least about 95% or 100% sequence identity with SEQ ID No. 10, 11, 12, or 13. In another embodiment, the V_{H} has an amino acid sequence having at least about 85%, at least about 90%, at least about 95% or 100% sequence identity with SEQ ID No. 14, 15, or 16.

In some embodiments, the anti-TROP2 antibody comprises a light chain (LC), wherein the LC has an amino acid sequence having at least about 85%, at least about 90%, at least about 95% or 100% sequence identity with SEQ ID No. 17, 18, 19, or 20. In some embodiments, the anti-TROP2 antibody comprises a heavy chain (HC). In an exemplary embodiment, the HC has an amino acid sequence having at least about 85%, at least about 90%, at least about 95% or 100% sequence identity with SEQ ID No. 21, 22, or 23.

In some embodiments, the anti-TROP2 antibody comprises a V_{L} and a V_{H}, wherein the V_{L} and the V_{H} have the amino acid sequences of SEQ ID No. 10 and SEQ ID No. 14; SEQ ID No. 11 and SEQ ID No. 14; SEQ ID No. 12 and SEQ ID No. 14; SEQ ID No. 12 and SEQ ID No. 15; SEQ ID No. 12 and SEQ ID No. 16; SEQ ID No. 13 and SEQ ID No. 14; SEQ ID No. 13 and SEQ ID No. 15; SEQ ID No. 10 and SEQ ID No. 15; SEQ ID No. 10 and SEQ ID No. 16; SEQ ID No. 11 and SEQ ID No. 15; SEQ ID No. 11 and SEQ ID No. 16; or SEQ ID No. 13 and SEQ ID No. 16, respectively.

In some embodiments, the anti-TROP2 antibody comprises a LC and HC, wherein the LC and HC have the amino acid sequences of SEQ ID No. 17 and SEQ ID No. 21; SEQ ID No. 18 and SEQ ID No. 21; SEQ ID No. 19 and SEQ ID No. 21; SEQ ID No. 19 and SEQ ID No. 22; SEQ ID No. 19 and SEQ ID No. 23; SEQ ID No. 20 and SEQ ID No. 21; SEQ ID No. 20 and SEQ ID No. 22; SEQ ID No. 17 and SEQ ID No. 22; SEQ ID No. 17 and SEQ ID No. 23; SEQ ID No. 18 and SEQ ID No. 22; SEQ ID No. 18 and SEQ ID No. 23; or SEQ ID No. 20 and SEQ ID No. 23, respectively.

Provided is also a nucleic acid encoding the anti-TROP2 antibody or antigen-binding fragment described herein or a vector comprising said nucleic acid. In some embodiments, the nucleic acids could be cloned into one or more vectors. In a preferred embodiment, said vector is an expression vector. Also provided herein is a host cell transformed with at least one nucleic acid or vector as described herein.

In another aspect, provided is an antibody-drug conjugate (ADC), comprising an antibody and a therapeutic agent conjugated through a linker, wherein the linker is selected from LU102, LU104 and LU104′, which have the following structures:

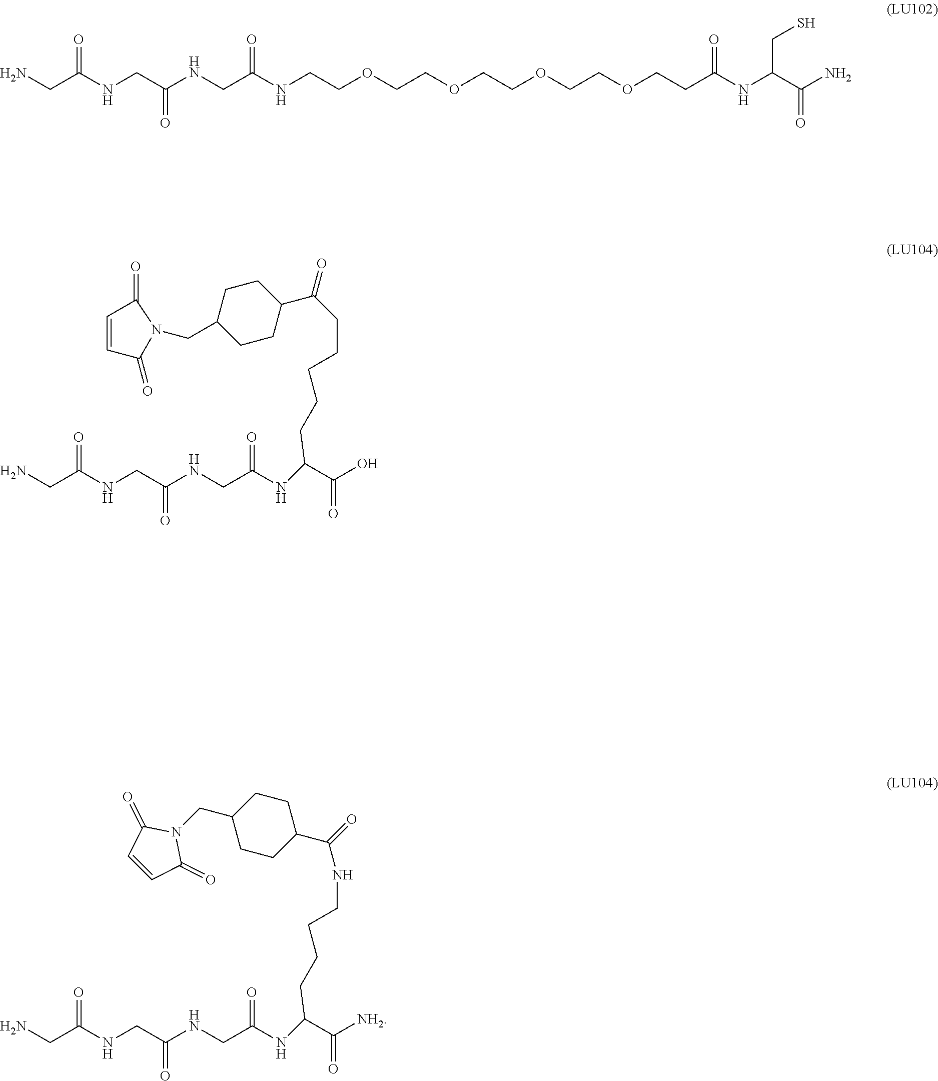 [SMILES: NCC(=O)NCC(=O)NCC(=O)NCCOCCOCCOCCOCCC(=O)NC(CS)C(N)=O ; NCC(=O)NCC(=O)NCC(=O)NC(CCCCCC(=O)C1CCC(CN2C(=O)C=CC2=O)CC1)C(=O)O ; NCC(=O)NCC(=O)NCC(=O)NC(CCCCNC(=O)C1CCC(CN2C(=O)C=CC2=O)CC1)C(N)=O]

In some embodiments, the ADC according to the present disclosure has the structure of Formula (1) or Formula (1′) or is a mixture thereof:

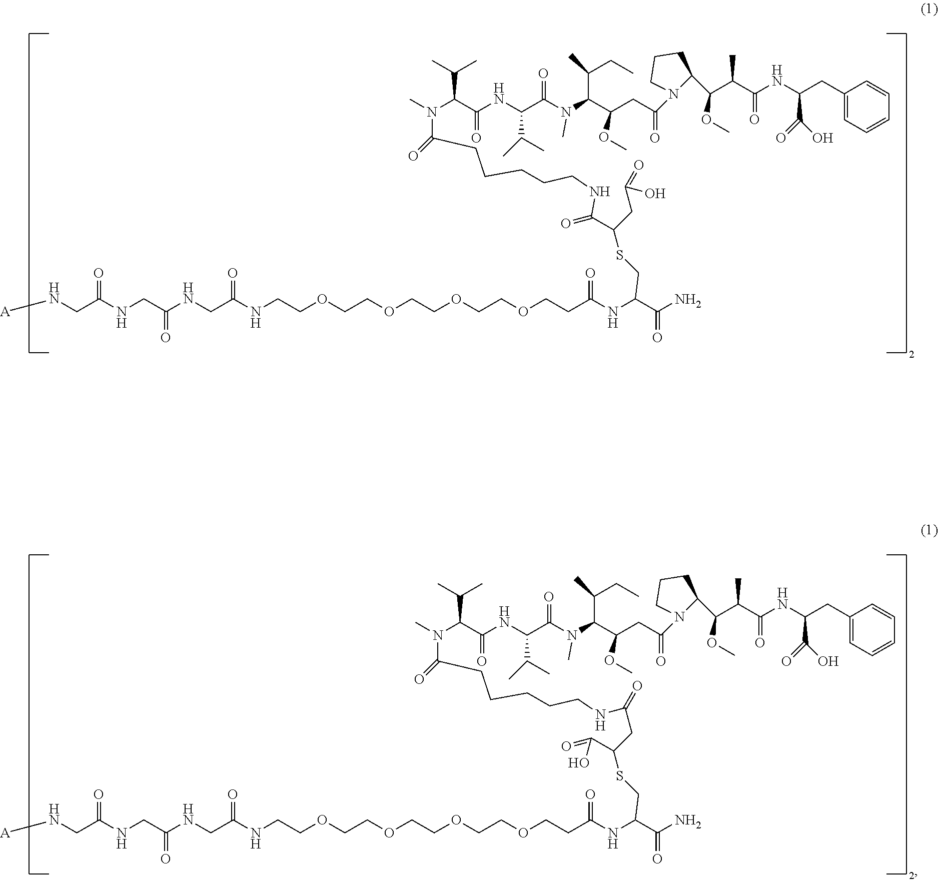

wherein A is an antibody, e.g., an antibody to TROP2; preferably, the antibody is selected from Ab0052, Ab0053, Ab0054, Ab0061, Ab0062, Ab0063, Ab0064 and Ab0002.

In some other embodiments, the ADC according to the present disclosure has the structure of Formula (2) or Formula (2′) or is a mixture thereof:

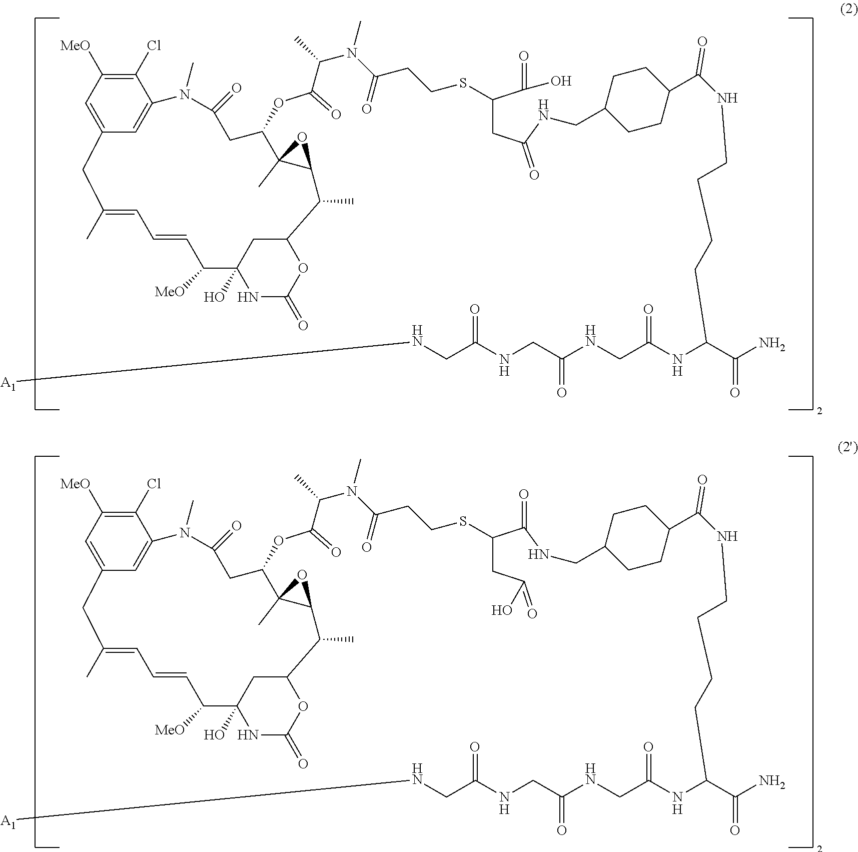

wherein A1 is an antibody, e.g., an antibody to TROP2; preferably, the antibody is selected from Ab0052, Ab0053, Ab0054, Ab0061, Ab0062, Ab0063, Ab0064 and Ab0002.

Also provided is an antibody-drug conjugate (ADC) comprising an antibody or antigen-binding fragment described herein conjugated with at least one therapeutic agent.

In another aspect, provided is a pharmaceutical composition, comprising the antibody or antigen-binding fragment thereof as described herein, the therapeutic nucleic acid or expression vector encoding the same, the antibody-drug conjugate, and/or the immune cell expressing a TROP2-targeting CAR.

In yet another aspect, provided is a method of treating a subject with a disease characterized by TROP2-positive diseased cells, wherein the method comprises administering to the subject in need thereof a therapeutically effective amount of the antibody or antigen-binding fragment thereof, the nucleic acid or expression vector, the antibody-drug conjugate, or the immune cell expressing a TROP2-targeting CAR, or the pharmaceutical composition as described herein.

### BRIEF DESCRIPTION OF THE DRAWINGS

FIG. 1 depicts V_{L} or V_{H} sequences of the human germline antibodies.

FIG. 2 depicts (a) Sequence alignment of the humanized V_{L} regions of the present disclosure, and (b) Sequence alignment of the humanized V_{H} regions of the present disclosure

FIG. 3 depicts in vivo tumor inhibition efficacy of ADCs DG1002 and DG1004.

FIG. 4 depicts in vivo tumor inhibition efficacy of ADC DG202.

### DETAILED DESCRIPTION

### Definitions

Unless defined otherwise, all technical and scientific terms used herein have the same meaning as is commonly understood by one of skill in the art. In addition, the terms and experimental procedures relating to protein and nucleic acid chemistry, molecular biology, cell and tissue culture, microbiology and immunology are those terms and common procedures widely used in the art. Meanwhile, for better understanding of the present disclosure, definitions and explanations of relevant terms are provided below.

As used herein, the expression “at least one” or “one or more” refers to one, two, three, four, five, six or more. As used herein, “a” and “an” unless clearly indicated to the contrary, should be understood to mean “at least one”.

As used herein, the term “antigen (Ag)” is any substance which could trigger immune response in the body, especially the production of antibodies. As used herein, this term refers to any substance comprising epitope(s) (i.e., antigenic determinant(s)) to which an antibody specifically binds. An antigen can be a polypeptide, lipid, carbohydrate, polynucleotide, etc. In a specific embodiment, the antigen refers to TROP2 proteins or fragments thereof comprising the epitope to which the antibody or antigen-binding fragment of the present disclosure binds. The TROP2 proteins or fragments thereof may be of any species, such as human, murine and rabbit or the like.

As used herein, the term “antibody (Ab)” is an immunoglobulin (Ig) molecule or fragment thereof that specifically binds to the epitope of an antigen through at least one antigen-binding site (i.e., the paratope). A “conventional” or “full-length” antibody typically consists of four polypeptides: two heavy chains (HC) and two light chains (LC). Each light chain, from the amino-terminus to the carboxyl-terminus, comprises a light chain variable region (V_{L}) and a light chain constant region (C_{L}). Each heavy chain, from the amino-terminus to the carboxyl-terminus, comprises a heavy chain variable region (V_{H}) and one or more heavy chain constant regions (C_{H}), which could include C_{H}1, C_{H}2, C_{H}3 and C_{H}4. When digested by the protease papain, a full-length antibody can be cleaved into two Fab fragments (i.e., the fragment antigen binding) and one Fc region (i.e., the fragment crystallizable region). Each Fab fragment essentially comprises a light chain and one variable and one constant region of the heavy chain (i.e., the V_{H}-C_{H}1, also known as the fragment difficult (Fd)), wherein the V_{L} and V_{H} form the antigen-binding site (also known as the fragment variable region, Fv), and the C_{L} and C_{H}1 are covalently linked by a disulfide bond. The V_{L} and V_{H} can be covalently connected by a polypeptide linker to form a single-chain Fv fragment (scFv). The Fv or the scFv can be further stabilized by introducing an interchain disulfide bond between the V_{L} and the V_{H} to generate a disulfide-stabilized variable fragment (dsFv) or a single-chain disulfide-stabilized variable fragment (scdsFv, with a linker peptide and an interchain disulfide bond), respectively. A single-chain Fab (scFab) fragment is generated by connecting the light chain and the Fd fragment with a polypeptide linker. The Fc region essentially comprises the remaining heavy chain constant regions (i.e., C_{H}2-C_{H}3 or C_{H}2-C_{H}3-C_{H}4) of the two heavy chains, covalently-linked by disulfide bonds at the hinge region. Another protease, pepsin, cleaves the antibody to produce an F(ab′)₂ fragment. The F(ab′)₂ fragment essentially contains two Fab fragments joined at the hinge region through disulfide bonds. An Fab′ fragment is one half of a F(ab′)₂ fragment, formed by the reduction of the disulfide bonds at the hinge region of the F(ab′)₂ fragment.

Each V_{L} and V_{H} has three hypervariable “complementarity-determining regions (CDRs)” and four relatively conserved “framework regions (FRs)”, arranged from the amino-terminus to the carboxyl-terminus in the following order: FR1, CDR1, FR2, CDR2, FR3, CDR3, FR4. One of skill in the art knows and can identify the CDRs and FRs using methodology of the art, for example, the Kabat or Chothia numbering (see e.g., Kabat, E. A. et al. (1991) Sequences of Proteins of Immunological Interest, Fifth Edition, U.S. Department of Health and Human Services, NIH Publication No. 91-3242, and Chothia, C. et al. (1987) J Mol. Biol. 196:901-917). Each CDR forms a CDR loop and contributes to the antigen-binding specificity and affinity. The framework residues form a scaffold and maintain the overall structure of the Fv domain. Some critical framework residues, referred to as “vernier zone residues”, usually identified during the design of humanized antibodies, are important for the antibody affinity and often kept during the antibody humanization process. The six CDR loops (i.e., three CDRs from each of V_{L} and V_{H}), with the help of the FRs, form a three-dimensional structure, which defines the paratope or the antigen-binding site of the antibody. According to the locations in the V_{L} or V_{H}, the CDR is designated as the heavy chain variable region CDR (CDR-H), like CDR-H1, CDR-H2, CDR-H3; or the light chain variable region CDR (CDR-L), like CDR-L1, CDR-L2, CDR-L3.

As used herein, the “affinity” with respect to an antibody or antigen-binding fragment is a measure of the strength with which the paratope of the antibody or antigen-binding fragment binds to an epitope (i.e., the cognate antigen). Typically, the antibody affinity is usually measured and reported by the equilibrium dissociation constant K_{D}. K_{D} can be calculated from kinetic analysis using the following equation:

K_{D}=Kd/Ka

wherein Kd and Ka are the dissociation and association rate constants between the antigen and the antibody, respectively.

The K_{D} and affinity are inversely related, that is, the smaller the K_{D} value, the greater the affinity. The typical K_{D} value for an antibody or antigen-binding fragment is in the range of μM (10⁻⁶M) to nM (10⁻⁷ to 10⁻⁹ M). The Kd and Ka can be measured using standard kinetic analysis methods, including, but not limited to, immunoassays, surface plasmon resonance (SPR) (Rich and Myszka (2000) Curr. Opin. Biotechnol 11:54; Englebienne (1998) Analyst. 123:1599), isothermal titration calorimetry (ITC) or other kinetic interaction assays known in the art (see, e.g., Paul, W. E., Fundamental Immunology, 2^{nd} ed., Raven Press, New York, pages 332-336 (1989); see also U.S. Pat. No. 7,229,619). Instrumentation and methods for real time detection and monitoring of binding rates are known and are commercially available (e.g., BiaCore 2000, Biacore AB, Upsala, Sweden).

As used herein, “specifically bind” refers to the affinity between an antibody or antigen-binding fragment and its cognate antigen. Generally, an antibody specifically binds to its cognate antigen with a high affinity, or a K_{D} value of 10⁻⁷ to 10⁻⁹ M or less, preferably 10⁻⁸ or less, 10⁻⁹ or less, or 10⁻¹⁰ or less.

As used herein, “thermal stability” is a measure of the ability of a biomolecule to maintain its structure/function when challenged by heat. Thermal stability is usually used to evaluate the biophysical quality of biomolecules. Therapeutic biomolecules (e.g., therapeutic antibodies) often benefit from favorable thermal stability, for example, extending shelf life and serum half-life, reducing the need for cold-chain storage, expanding the range of applications for practical use. The thermal stability is usually reported as the unfolding midpoint or the midpoint of hydrophobic exposure Tₘ, which is the temperature at which the populations of folded and unfolded biomolecules are identical. Tₘ could be measured in a thermal shift assay using well-known methods in the art, for example, the differential scanning calorimetry (DSC) or differential scanning fluorimetry (DSF). A typical Tₘ of an antibody could be about 60° C. or above, 65° C. or above, 70° C. or above, or 75° C. or above. Generally, such Tₘ value is about 95° C. or lower, for example about 90° C. or lower or about 88° C. or lower.

As used herein, the definition of “antibody” encompasses conventional antibodies, recombinant antibodies, multispecific antibodies (e.g., bispecific antibodies), fully human antibodies, non-human antibodies, humanized antibodies, chimeric antibodies, intrabodies, diabodies, anti-idiotypic antibodies, and antigen-binding fragments. Antibodies provided herein include members of any immunoglobulin type (e.g., IgG, IgM, IgD, IgE, IgA and IgY), any class (e.g. IgG1, IgG2, IgG3, IgG4, IgA1 and IgA2) or subclass (e.g., IgG2a and IgG2b).

As used herein, a “chimeric antibody” is an antibody comprising non-human variable regions and human constant regions. Techniques for producing a chimeric antibody are well known in the art (see, e.g., Jones et al., 1986, Nature, 321:522; Sandhu, Crit. Rev. Biotech., 1992, 12:437.)

As used herein, a “humanized antibody” is an antibody derived from a non-human antibody whose sequence has been modified to increase the sequence similarity to human immunoglobulin molecules. A humanized antibody has a lower immunogenicity inside the human body compared to its non-human counterpart. A chimeric antibody can be humanized using well known techniques in the art (see, e.g., Morrison et al. (1984) Proc. Natl. Acad. Sci. 81(21): 6851-6855; Neuberger et al. (1984) Nature 312: 604-608).

As used herein, an “antigen-binding fragment” of an antibody refers to any portion of a full-length antibody that contains at least a portion of the variable domains (e.g. one or more CDRs) of the antibody and specifically binds to the same cognate antigen as the full-length antibody. The antigen-binding fragment as described herein can be produced by enzymatic treatment of full-length antibodies and/or synthetic methods, such as recombinant antibodies produced by grafting the CDRs of an antibody into the framework of another antibody. Examples of antigen-binding fragments include, but are not limited to, Fv, scFv, dsFv, scdsFv, diabody, Fab, scFab, Fab′, F(ab′)₂, and other fragments. An antigen-binding fragment generally contains at least or about 50 amino acids and typically at least or about 200 amino acids.

As used herein, an “antibody” or “antigen-binding fragment” comprises at least one antigen-binding site or a paratope. Therefore, the definition of “antibody” or “antigen-binding fragment” encompasses any variant derived from any antibody or any antigen-binding fragment as described above, such as amino acid sequence variants, glycosylation variants, and covalently modified variants.

As used herein, “amino acid sequence variants” of a protein can be generated through substituting one or more amino acid residues with corresponding conservative residues without affecting the biological activity of the protein. Suitable conservative substitutions of amino acids are known to those of skill in this art. Those of skill in this art recognize that, in general, single amino acid substitutions in non-essential regions of a polypeptide do not substantially alter its biological activity (see, e.g., Watson et al., Molecular Biology of the Gene, 4^{th} Edition, 1987, The Benjamin/Cummings Pub. Co., p. 224).

As used herein, “glycosylation” refers to a post-translational modification of a protein leading to the addition of carbohydrate or glycan moieties to the protein backbone (e.g., serine- or threonine-linked glycosylation for O-linked glycans and asparagine-linked glycosylation for N-linked glycans). Those of skill in the art are able to generate glycosylation variants of a therapeutic antibody to achieve desired therapeutic efficacies using techniques known in the art. As used herein, “covalently modified variants” of an antibody or antigen-binding fragment can be generated by introducing natural or unnatural amino acids, compounds, or peptide linkers through covalently linkage. Hence, an antibody or antigen-binding fragment includes any protein having an antigen-binding domain that is homologous or substantially homologous to an immunoglobulin antigen-binding domain.

As used herein, “polypeptide” refers to two or more amino acids covalently joined. The terms “polypeptide” and “protein” are used interchangeably herein.

A “host cell” may be any prokaryotic or eukaryotic cell that contains exogenous polynucleotides.

As used herein, a “nucleic acid” or a “polynucleotide” refers to a polymer of at least two nucleotides or nucleotide derivatives joined together by phosphodiester bonds, including deoxyribonucleic acid (DNA) and ribonucleic acid (RNA). Exogenous nucleic acids can be introduced into a host cell in the form of vectors. Therapeutic nucleic acids can be introduced into a subject with a purpose of treating a disease. As used herein, therapeutic nucleic acids, such as vectors, comprise the nucleic acids encoding any anti-TROP2 antibody or antigen-binding fragment as described herein.

As used herein, the term “expression” refers to the production of RNA polynucleotides from transcription or polypeptides from translation. The expression level of a polypeptide in a biological sample can be examined using any method known in art. Such methods include, but are not limited to, enzyme-linked immunosorbent assay (ELISA), western blot, flow cytometry, immunofluorescence imaging and immunohistochemistry using antibodies that specifically bind to the polypeptide to be examined.

As used herein, a “vector” is a vehicle used to transfer exogenous nucleic acids into a host cell, where the exogenous nucleic acids are amplified or expressed. As used herein, the definition of “vector” encompasses plasmids, linearized plasmids, viral vectors, cosmids, phage vectors, phagemids, artificial chromosomes (e.g., yeast artificial chromosomes and mammalian artificial chromosomes), etc. As used herein, a vector could be expressible and/or replicable inside a host cell, meaning that the vector is able to express RNA polynucleotides or polypeptides and/or to produce multiple copies of the vector in the host cell. To be “expressible” or “replicable”, a vector could comprise nucleic acid sequences or elements operably linked to a promoter. As used herein, “operably linked” with reference to nucleic acid sequences or elements means that these nucleic acid sequences are functionally related to each other. For example, a promoter can be operably linked to a nucleic acid sequence encoding a polypeptide, whereby the promoter regulates or mediates the transcription of the nucleic acid. Those skilled in the art could select and use appropriate vectors for a particular purpose.

As used herein, the term “payload” refers to a functional moiety which is comprised in the conjugate according to the present disclosure, for example, linked via a linker. The payload may be selected from the group consisting of small molecule compounds, nucleic acids and analogues, tracer molecules (including fluorescent molecules, etc.), short peptides, polypeptides, peptidomimetics, and proteins. In one embodiment, the payload is selected from the group consisting of small molecule compounds, nucleic acid molecules, and tracer molecules. In a preferred embodiment, the payload is selected from small molecule compounds. In a more preferred embodiment, the payload is selected from the group consisting of cytotoxin and fragments thereof.

A non-limiting example of payload may be a therapeutic agent. A “therapeutic agent” refers to a substance with a therapeutic effect when applied to a subject. Examples of therapeutic agents include, but are not limited to, small-molecule drugs which are usually cytotoxins (e.g., Mertansine (DM1), monomethyl auristatin F (MMAF), monomethyl auristatin D (MMAD) and monomethyl auristatin E (MMAE)), oligonucleotides and analogs thereof (e.g., interfering RNAs), biologically active peptides (e.g., Glucagon-like Peptide-1), therapeutic antibodies, protein toxins (e.g., diphtheria toxin) and enzymes (e.g., urease).

A “cytotoxin” refers to a substance that inhibits or prevents the expression activity of a cell, cellular function, and/or causes destruction of cells. Examples of cytotoxins include, but are not limited to, drugs that target the following targets: the microtubule cytoskeleton, DNA structure or DNA synthesis or repair, RNA synthesis, protein synthesis, kinesin-mediated protein transport, apoptosis. Drugs targeting the microtubule cytoskeleton may be, for example, a microtubule stabilizer or a tubulin polymerization inhibitor. Examples of microtubule stabilizers include, but are not limited to, taxanes. Examples of tubulin polymerization inhibitors include, but are not limited to, maytansinoids, auristatins, vinblastines, colchicines, and dolastatins. The DNA-targeting drug can be, for example, a drug or a topoisomerase inhibitor that directly disrupts the DNA structure, such as DNA double strand breakers, DNA alkylating agents, DNA intercalators. The DNA double-strand disrupting agent can be, for example, an enediyne antibiotic, including but not limited to dynemicin, esperamicin, neocarzinostatin, uncialamycin, and the like. The DNA alkylating agent may be, for example, a DNA bis-alkylator (i.e. DNA-cross linker) or a DNA mono-alkylator. Examples of DNA alkylating agents include but are not limited to pyrrolo[2,1-c][1,4]benzodiazepine (PBD) dimer, 1-(chloromethyl)-2,3-dihydrogen-1H-benzo[e]indole (CBI) dimer, CBI-PBD heterodimer, dihydroindolobenzodiazepine (IGN) dimer, duocarmycin-like compound, and the like. Examples of topoisomerase inhibitors include but are not limited to camptothecins and anthracyclines. The RNA-targeting drug may be, for example, a drug that inhibits splicing, and examples thereof include but are not limited to pladienolide. Drugs that target kinesin-mediated protein transport can be, for example, mitotic kinesin inhibitors including, but not limited to, kinesin spindle protein (KSP) inhibitors.

A spacer is a structure that is located between different structural modules and can spatially separate the structural modules. The definition of spacer is not limited by whether it has a certain function or whether it is cleavable or degradable. Examples of spacers include, but are not limited to, amino acids and non-amino acid structures, wherein non-amino acid structures can be, but are not limited to, amino acid derivatives or analogues. A “Spacer sequence” refers to an amino acid sequence serving as a spacer, and examples thereof include but are not limited to a single amino acid such as Leu, Gln, etc., a sequence containing a plurality of amino acids, for example, a sequence containing two amino acids such as GA, etc., or, for example, GGGS, GGGGSGGGGS, etc. Other examples of spacers include, for example, self-immolative spacers such as PAB (p-aminobenzyl), and the like.

As used herein, a “Sortase recognition motif” or “Sortase donor motif” refers to the amino acid sequence recognized by a Sortase enzyme that enables conjugation with a Sortase acceptor motif. Examples of Sortase acceptor motifs are not limited to N-terminal oligomeric glycine, oligomeric alanine, and oligomeric glycine/alanine having a degree of polymerization of 3-10. For example, an oligomeric glycine motif can be a Gₙ motif, wherein G is Gly, n is an integer of 3 to 10. The Sortase recognition motif corresponds to the type of sortase enzyme. For example, the Sortase recognition motif for Sortase A from Staphylococcus aureus or Streptococcus pyogenes can be LPXTGJ, wherein L is Leu, P is Pro, X is any natural or unnatural amino acid, T is Thr, G is Gly, and J is absent or an amino acid fragment comprising 1-10 amino acids. A moiety comprising a Sortase recognition motif (such as the antibody according to the present disclosure) can be conjugated to a moiety comprising a Sortase acceptor motif (e.g., the linker-payload intermediate obtained from a linker (such as LU102, LU104 or LU104′) and a therapeutic agent as described herein under the catalysis of the corresponding Sortase. For example, in a Sortase A-mediated conjugation reaction (such as Sortase A from Staphylococcus aureus or Streptococcus pyogenes), the peptide bond between the Thr and Gly in the LPXTGJ motif comprised by a first moiety (e.g., an antibody comprising a LPETGG motif at the C-terminal of the light chain) is cleaved by the Sortase A, the resulting intermediate is linked to the free N-terminal of Gₙ comprised by a second moiety (e.g., a linker-payload intermediate comprising a Gₙ, such as IM102 or IM104 as described herein) through formation of a new Thr-Gly peptide bond, and therefore the two moieties are linked through the formation of LPXTGₙ. Gₙ and LPXTGJ are as defined above.

As used herein, “sequence identity” has an art-recognized meaning and the percent of sequence identity between two nucleic acids or polypeptides can be calculated by aligning the two sequences using published algorithms, such as the Basic Local Alignment Search Tool (BLAST) and the Fast Adaptive Shrinkage/Thresholding Algorithm (FASTA) (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994). While there are a number of methods to measure identity between two polynucleotides or polypeptides, the term “identity” is well known to skilled artisans (Carrillo, H. & Lipman, D., SIAM J Applied Math 48:1073 (1988)).

As used herein, “treating” a subject with a disease or condition means that administering or applying a composition, a procedure or a regimen to the subject in an attempt to cure the disease or condition, or to arrest, alleviate, ameliorate or eliminate the symptoms of the disease or condition. Hence treatment encompasses prophylaxis, therapy and/or cure. “Prophylaxis” refers to prevention of a potential disease and/or a prevention of worsening of symptoms or progression of a disease. As used herein, treatment also encompasses any pharmaceutical use of any antibody or antigen-binding fragment thereof or pharmaceutical compositions as provided herein. Non-limiting examples of the disease include but not limited to TROP2-positive cancers, preferably epithelial cancers, including, but not limited to, gastric cancer, breast cancer, urothelial cancer, lung cancer, liver cancer, endometrial cancer, head and neck cancer, and ovarian cancer.

As used herein, the definition of “subject” includes human and non-human subjects, such as experimental animals (e.g., mouse, rabbit, rat and non-human primates), and preferably human.

As used herein, a “therapeutic effect” means an effect resulting from treatment of a subject that alters, typically ameliorates or eliminates the symptoms of a disease or condition.

As used herein, a “therapeutically effective amount” refers to the quantity of an agent, compound, or composition containing one or more active agents that is at least sufficient to produce a therapeutic effect following administration to a subject. Hence, it is the quantity necessary for preventing, curing, ameliorating, arresting or partially arresting a symptom of a disease or disorder.

In the context of the present specification, the positions of amino acids in the V_{L} are defined as follows: (i) based on any one of SEQ ID Nos 10-13, as they have identical length; (ii) starting from the N-terminus; (iii) the position of the 1^{st} amino acid from the N-terminus is designated as 1. The positions of amino acids in the V_{H} are defined similarly based on any one of SEQ ID Nos 14-16 as they have identical length.

### Anti-TROP2 Antibody or Antigen-Binding Fragment Thereof

In an aspect, provided is an anti-TROP2 antibody or antigen-binding fragment thereof, comprising a light chain variable region (V_{L}) wherein the V_{L} has CDR-L1, CDR-L2 and CDR-L3.

In an embodiment according to the present disclosure, the V_{L} comprises:

(i) CDR-L1 having an amino acid sequence of SEQ ID No. 1,

(ii) CDR-L2 having an amino acid sequence of SEQ ID No. 2 or SEQ ID No. 3, and

(iii) CDR-L3 having an amino acid sequence of SEQ ID No. 4.

In another embodiment according to the present disclosure, the V_{L} has an amino acid sequence having at least about 85%, at least about 90%, at least about 95% or 100% sequence identity with SEQ ID No. 10, 11, 12, or 13. In some embodiments, the V_{L} has an amino acid sequence having about 90%, about 91%, about 92%, about 93%, about 94%, about 95%, about 96%, about 97%, about 98%, about 99% or 100% sequence identity with SEQ ID No. 10, 11, 12, or 13. In a preferred embodiment, the V_{L} has the amino acid sequence of SEQ ID No. 10, 11, 12, or 13.

In some embodiments, the anti-TROP2 antibody according to the present disclosure comprises a light chain (LC). In an exemplary embodiment, the LC has an amino acid sequence having at least about 85%, at least about 90%, at least about 95% or 100% sequence identity with SEQ ID No. 17, 18, 19, or 20. In some embodiments, the LC has an amino acid sequence having about 90%, about 91%, about 92%, about 93%, about 94%, about 95%, about 96%, about 97%, about 98%, about 99%, or 100% sequence identity with SEQ ID No. 17, 18, 19, or 20. In a preferred embodiment, the LC has the amino acid sequence of SEQ ID No. 17, 18, 19, or 20.

The anti-TROP2 antibody according to the present disclosure comprises a heavy chain variable region (V_{H}), wherein the V_{H} comprises:

(i) CDR-H1 having an amino acid sequence of SEQ ID No. 5,

(ii) CDR-H2 having an amino acid sequence of SEQ ID No. 6, SEQ ID No. 7 or SEQ ID No. 8, and

(iii) CDR-H3 having an amino acid sequence of SEQ ID No. 9.

In another embodiment according to the present disclosure, the V_{H} has an amino acid sequence having at least about 85%, at least about 90%, at least about 95% or 100% sequence identity with SEQ ID No. 14, 15, or 16. In some embodiments, the V_{H} has an amino acid sequence having about 90%, about 91%, about 92%, about 93%, about 94%, about 95%, about 96%, about 97%, about 98%, about 99% or 100% sequence identity with SEQ ID No. 14, 15, or 16. In a preferred embodiment, the V_{H} has the amino acid sequence of SEQ ID No. 14, 15, or 16.

In some embodiments, the anti-TROP2 antibody according to the present disclosure comprises a heavy chain (HC). In an exemplary embodiment, the HC has an amino acid sequence having at least about 85%, at least about 90%, at least about 95% or 100% sequence identity with SEQ ID No. 21, 22, or 23. In some embodiments, the HC has an amino acid sequence having about 90%, about 91%, about 92%, about 93%, about 94%, about 95%, about 96%, about 97%, about 98%, about 99%, or 100% sequence identity with SEQ ID No. 21, 22, or 23. In a preferred embodiment, the HC has the amino acid sequence of SEQ ID No. 21, 22, or 23.

In a specific embodiment, the anti-TROP2 antibody according to the present disclosure comprises a V_{L} and a V_{H}, wherein the V_{L} and a V_{H} are as defined above.

In a specific embodiment, the anti-TROP2 antibody according to the present disclosure comprises a LC and a HC, wherein the LC and HC are as defined above.

In some embodiments, the anti-TROP2 antibody according to the present disclosure comprises a V_{L} comprising a CDR-L1 having an amino acid sequence of SEQ ID No. 1, a CDR-L2 having an amino acid sequence of SEQ ID No. 2 and a CDR-L3 having an amino acid sequence of SEQ ID No. 4; and a V_{H} comprising a CDR-H1 having an amino acid sequence of SEQ ID No. 5, a CDR-H2 having an amino acid sequence of SEQ ID No. 6 and a CDR-H3 having an amino acid sequence of SEQ ID No. 9.

In some embodiments, the anti-TROP2 antibody according to the present disclosure comprises a V_{L} comprising a CDR-L1 having an amino acid sequence of SEQ ID No. 1, a CDR-L2 having an amino acid sequence of SEQ ID No. 2 and a CDR-L3 having an amino acid sequence of SEQ ID No. 4; and a V_{H} comprising a CDR-H1 having an amino acid sequence of SEQ ID No. 5, a CDR-H2 having an amino acid sequence of SEQ ID No. 7 and a CDR-H3 having an amino acid sequence of SEQ ID No. 9.

In some embodiments, the anti-TROP2 antibody according to the present disclosure comprises a V_{L} comprising a CDR-L1 having an amino acid sequence of SEQ ID No. 1, a CDR-L2 having an amino acid sequence of SEQ ID No. 2 and a CDR-L3 having an amino acid sequence of SEQ ID No. 4; and a V_{H} comprising a CDR-H1 having an amino acid sequence of SEQ ID No. 5, a CDR-H2 having an amino acid sequence of SEQ ID No. 8 and a CDR-H3 having an amino acid sequence of SEQ ID No. 9.

In some embodiments, the anti-TROP2 antibody according to the present disclosure comprises a V_{L} comprising a CDR-L1 having an amino acid sequence of SEQ ID No. 1, a CDR-L2 having an amino acid sequence of SEQ ID No. 3 and a CDR-L3 having an amino acid sequence of SEQ ID No. 4; and a V_{H} comprising a CDR-H1 having an amino acid sequence of SEQ ID No. 5, a CDR-H2 having an amino acid sequence of SEQ ID No. 6 and a CDR-H3 having an amino acid sequence of SEQ ID No. 9.

In some embodiments, the anti-TROP2 antibody according to the present disclosure comprises a V_{L} comprising a CDR-L1 having an amino acid sequence of SEQ ID No. 1, a CDR-L2 having an amino acid sequence of SEQ ID No. 3 and a CDR-L3 having an amino acid sequence of SEQ ID No. 4; and a V_{H} comprising a CDR-H1 having an amino acid sequence of SEQ ID No. 5, a CDR-H2 having an amino acid sequence of SEQ ID No. 7 and a CDR-H3 having an amino acid sequence of SEQ ID No. 9.

In some embodiments, the anti-TROP2 antibody according to the present disclosure comprises a V_{L} comprising a CDR-L1 having an amino acid sequence of SEQ ID No. 1, a CDR-L2 having an amino acid sequence of SEQ ID No. 3 and a CDR-L3 having an amino acid sequence of SEQ ID No. 4; and a V_{H} comprising a CDR-H1 having an amino acid sequence of SEQ ID No. 5, a CDR-H2 having an amino acid sequence of SEQ ID No. 8 and a CDR-H3 having an amino acid sequence of SEQ ID No. 9.

In a more specific embodiment, the anti-TROP2 antibody according to the present disclosure comprises a V_{L} and a V_{H}, wherein the V_{L} and the V_{H} have the amino acid sequences of SEQ ID No. 10 and SEQ ID No. 14 (corresponding to Ab0052); SEQ ID No. 11 and SEQ ID No. 14 (corresponding to Ab0053); SEQ ID No. 12 and SEQ ID No. 14 (corresponding to Ab0054); SEQ ID No. 12 and SEQ ID No. 15 (corresponding to Ab0061); SEQ ID No. 12 and SEQ ID No. 16 (corresponding to Ab0062); SEQ ID No. 13 and SEQ ID No. 14 (corresponding to Ab0063); SEQ ID No. 13 and SEQ ID No. 15 (corresponding to Ab0064); SEQ ID No. 10 and SEQ ID No. 15; SEQ ID No. 10 and SEQ ID No. 16; SEQ ID No. 11 and SEQ ID No. 15; SEQ ID No. 11 and SEQ ID No. 16; or SEQ ID No. 13 and SEQ ID No. 16, respectively.

In a more specific embodiment, the anti-TROP2 antibody according to the present disclosure comprises a LC and HC, wherein the LC and HC have the amino acid sequences of SEQ ID No. 17 (or SEQ ID No. 27) and SEQ ID No. 21 (corresponding to Ab0052); SEQ ID No. 18 (or SEQ ID No. 28) and SEQ ID No. 21 (corresponding to Ab0053); SEQ ID No. 19 (or SEQ ID No. 29) and SEQ ID No. 21 (corresponding to Ab0054); SEQ ID No. 19 (or SEQ ID No. 29) and SEQ ID No. 22 (corresponding to Ab0061); SEQ ID No. 19 (or SEQ ID No. 29) and SEQ ID No. 23 (corresponding to Ab0062); SEQ ID No. 20 (or SEQ ID No. 30) and SEQ ID No. 21 (corresponding to Ab0063); SEQ ID No. 20 (or SEQ ID No. 30) and SEQ ID No. 22 (corresponding to Ab0064); SEQ ID No. 17 (or SEQ ID No. 27) and SEQ ID No. 22; SEQ ID No. 17 (or SEQ ID No. 27) and SEQ ID No. 23; SEQ ID No. 18 (or SEQ ID No. 28) and SEQ ID No. 22; SEQ ID No. 18 (or SEQ ID No. 28) and SEQ ID No. 23; or SEQ ID No. 20 (or SEQ ID No. 30) and SEQ ID No. 23, respectively.

In a specific embodiment, antibodies according to the present disclosure are as shown in Table 1.

TABLE 1

| Anti- | CDR- | CDR- | CDR- | CDR- | CDR- | CDR- | | | | |
|---|---|---|---|---|---|---|---|---|---|---|
| body | LI | L2 | L3 | Hl | H2 | H3 | V_{L} | V_{H} | LC | HC |
| Ab0052 | 1 | 2 | 4 | 5 | 6 | 9 | 10 | 14 | 17 | 21 |
| | | | | | | | | | (or 27) | |
| Ab0053 | 1 | 2 | 4 | 5 | 6 | 9 | 11 | 14 | 18 | 21 |
| | | | | | | | | | (or 28) | |
| Ab0054 | 1 | 2 | 4 | 5 | 6 | 9 | 12 | 14 | 19 | 21 |
| | | | | | | | | | (or 29) | |
| Ab0061 | 1 | 2 | 4 | 5 | 7 | 9 | 12 | 15 | 19 | 22 |
| | | | | | | | | | (or 29) | |
| Ab0062 | 1 | 2 | 4 | 5 | 8 | 9 | 12 | 16 | 19 | 23 |
| | | | | | | | | | (or 29) | |
| Ab0063 | 1 | 3 | 4 | 5 | 6 | 9 | 13 | 14 | 20 | 21 |
| | | | | | | | | | (or 30) | |
| Ab0064 | 1 | 3 | 4 | 5 | 7 | 9 | 13 | 15 | 20 | 22 |
| | | | | | | | | | (or 30) | |

Note: The numbers in the table refer to corresponding SEQ ID Nos, e.g. 10 refers to SEQ ID No 10.

In some preferable embodiments, the antibody according to the present disclosure is a full-length antibody. In some embodiments, the antibody is an antigen-binding fragment comprising at least one antigen-binding site of the full-length antibody. In some embodiments, the antibody of the present disclosure is IgG. In a preferred embodiment, the antibody is a humanized antibody.

Said antibody specifically binds to TROP2. In some embodiments, the TROP2 is a full-length protein or fragment thereof having an epitope to which the antibody according to the present disclosure binds. The TROP2 may be human or non-human TROP2, for example, rat, mouse, rabbit, and preferably human.

The antibody or antigen-binding fragment (e.g. the light chain or heavy chain) according to the present disclosure may be modified such as addition of one or more additional elements for incorporation into an ADC. Non-limiting examples of such elements can be active groups, natural or unnatural amino acids, peptides or combinations thereof.

Additional elements can be introduced to the antibody or antigen-binding fragment according to the present disclosure for various purposes. In some embodiments, the additional element is an active group such that the antibody or antigen-binding fragment can be conjugated with a payload through the active group to produce an antibody-drug conjugate. In some embodiments, the additional element is a peptide, such as a ligase recognition motif (e.g., a Sortase recognition motif (LPETGG)), a signal peptide or a spacer sequence (e.g., GA) described herein or a combination thereof.

The introduction position of additional elements is not limited, for example, can be, but not limited to, at the C-terminal or the N-terminal of the heavy chain or light chain of the antibody. In a particular embodiment, a Sortase recognition motif (LPETGG) and a spacer sequence (GA) (together as GALPETGG, corresponding to SEQ ID No. 25) may be added to the C-terminus of the light chain. In a particular embodiment, a Sortase recognition motif and a spacer sequence (together as GALPETGG, corresponding to SEQ ID No. 25) are introduced at the C-terminus of SEQ ID Nos. 27-30 to give SEQ ID Nos. 17-20, respectively. Accordingly, the antibody or antigen-binding fragment (e.g., the light chain or heavy chain), with or without such additional element, is encompassed within the scope of the present disclosure, and a person skilled in the art should understand, for example, when referring to the sequence of a light chain, it can refer to a sequence with or without the additional element like SEQ ID No. 25. For example, SEQ ID Nos. 27-30 are equivalent to SEQ ID Nos. 17-20, respectively, upon description of the light chain sequence.

Antibodies or antigen-binding fragments modified as above from the antibody or antigen-binding fragment according to the present disclosure are encompassed within the scope of the present disclosure.

### Nucleic Acid, Vector and Host Cell

In another aspect, provided is a nucleic acid encoding the anti-TROP2 antibody or antigen-binding fragment of the present disclosure. Also provided is a vector comprising said nucleic acid. In some embodiments, nucleic acids of the present disclosure could be cloned into one or more vectors. In some preferred embodiments, said vector is an expression vector, such as a plasmid for expression in bacteria, yeast or mammalian cells, or a phage vector and a phagemid vector for phage display.

Also provided is a host cell transformed with at least one nucleic acid or vector as described above. A host cell herein can be used to produce the anti-TROP2 antibody or antigen-binding fragment thereof of the present disclosure. Examples of host cells include, but are not limited to, prokaryotic cells such as bacteria, lower eukaryotic cells such as yeast, or higher eukaryotic cells such as mammalian cells.

Nucleic acids of the present disclosure can be obtained from various sources, for example, phage display libraries, yeast display libraries, hybridoma cells (e.g., mouse B cell hybridoma cells), and synthetic methods. Methods of obtaining nucleic acids of the present disclosure from said libraries or hybridoma cells are well known in the art.

In some embodiments, nucleic acids of the present disclosure are prepared as recombinant nucleic acids comprising additional nucleic acid sequences, such as regulatory elements and nucleic acid sequences encoding desired peptides or proteins. Said recombinant nucleic acids comprising nucleic acids of the present disclosure can be prepared using molecular cloning techniques well known in the art, for example, chemical synthesis, site-directed mutagenesis and polymerase chain reaction (PCR) techniques (see Sambrook, J., E. F. Fritsch, and T. Maniatis. (1989). Molecular cloning: a laboratory manual, 2nd ed. Cold Spring Harbor Laboratory, Cold Spring Harbor, N.Y.). Said regulatory elements could regulate the expression of the antibody or antigen-binding fragment, including, but not limited to, enhancers, insulators, internal ribosome entry sites (IRES). Said peptides or proteins could facilitate the detection or isolation of the expressed antibody or fragment, including, but not limited to, affinity tags (e.g., a biotin tag, a polyhistidine-tag (His₆) or a Glutathione S-Transferase (GSH) tag), enzyme-cleavable peptides, and reporter proteins (e.g., fluorescent proteins). In some embodiments, nucleic acids of the present disclosure and said regulatory elements and/or nucleic acid sequences encoding desired peptides or proteins can be operably linked to a promoter. Depending on the type of host cells and purification strategies to be used, those of skill in the art are able to select suitable expression vectors, promoters, regulatory elements, and peptides or proteins.

Also provided is a method of producing the anti-TROP2 antibody or antigen-binding fragment of the present disclosure in a host cell, wherein said method comprises the following steps:

(i) transforming the host cell with at least one nucleic acid or expression vector as described herein,

(ii) culturing the transformed host cell under suitable conditions to allow the expression of said nucleic acid or expression vectors, and optionally

(iii) isolating and purifying of the antibody or antigen-binding fragment of the present disclosure from the host cell or the culture medium.

Methods for transforming a host cell are well known in the art, including, but not limited to, calcium phosphate transfection, Diethylaminoethyl-dextran transfection, electroporation, liposome transfection, and phage infection (see e.g., Maniatis et al (1982) (Molecular Cloning, Cold Spring Harbor, N.Y.) and Primrose and Old (1980) (Principles of Gene Manipulation, Blackwell, Oxford).

The host cell can be stably or transiently transformed. In some embodiments, a single vector comprising the nucleic acids encoding both heavy and light chains could be used. In a particular embodiment, two vectors are used, wherein one vector encodes the light chain and the other encodes the heavy chain of said antibody. In some embodiments, the expression vector(s) could further contain one or more selective marker genes, for example, a neomycin or puromycin resistance gene. In some embodiments, chaperon plasmids may be introduced into the same host cell together with the expression vectors described above, for example, in bacteria, to assist the solubilisation and/or folding of the antibodies. Isolation and purification of antibodies may be performed using well-known techniques in the art, for example, using a Protein A affinity column.

Bacteria (e.g., E. coli. BL21(DE3)) are particularly advantageous for the expression of smaller antigen-binding fragments, for example, the Fv, scFvs, Fab and Fab′ fragments. In some embodiments, the nucleic acid sequences encoding the two variable domains are cloned into a single vector and introduced into E. coli. BL21(DE3). Mammalian host cells suitable for antibody expression include, but are not limited to, myeloma cells, Chinese Hamster Ovary (CHO) cells, and other mammalian cells suitable for expressing antibodies. In a particular embodiment, a mammalian host cell is transformed with two expression vectors, each encoding the heavy chain and light chain of the anti-TROP2 antibody, and the expressed antibodies are purified using a Protein A affinity column.

In some other embodiments, the anti-TROP2 antibody or antigen-binding fragment of the present disclosure may be isolated from a library, such as a yeast display library or a phage display library using techniques known in the art (see, e.g., Winter et al., (1994) Annu. Rev. Immunol. 12:433-455 and McCafferty et al., (1990) Nature 348:552-553).

### Humanization of Non-Human Antibodies

The antibody or antigen-binding fragment of the present disclosure can be produced from a non-human antibody, for example, a chimeric antibody. As an example, the preparation can be as follows. First, the V_{L} and V_{H} regions of the chimeric antibody are subjected to molecular modeling using well-established structures as templates. Template information can be obtained from a public database, for example, the Protein Data Bank (PDB). In parallel, the V_{L} and V_{H} sequences of the chimeric antibody are subjected to homology search against human antibody germline genes in an antibody gene database (e.g., the international ImMunoGeneTics information system (IMGT) or Igblast tool in the National Center for Biotechnology Information (NCBI)). Then human germline genes with highest sequence identity to the V_{L} and V_{H} sequences of the chimeric antibody are selected as framework (FR) donors. Finally, the CDRs of the chimeric antibody are grafted into the corresponding regions of the human germline genes. To preserve the stability and antigen specificity of the humanized antibody, back mutations may be introduced into the human FR regions, wherein some of the human FR residues are replaced with the counterpart residues in the chimeric antibody.

### Antibody-Drug Conjugates

Also provided is an antibody-drug conjugate (ADC) comprising an antibody or antigen-binding fragment of the present disclosure conjugated with at least one therapeutic agent. In some embodiments, the therapeutic agent and the antibody are linked, optionally covalently linked. Active groups available for covalent conjugation may be provided by side chains of the amino acid residues of the antibody or modified groups of the antibody, for example, amine, carboxyl, phenyl, thiol or hydroxyl groups. Preferably, the conjugation of therapeutic agents to the antibody or antigen-binding fragment does not interfere with the antibody-antigen binding. In some embodiments, the therapeutic agent and the antibody are (optionally covalently) linked by a linker. The linker can be cleavable or non-cleavable. Examples of cleavable linkers include, but are not limited to, enzyme-cleavable linkers (e.g., peptide linkers), acid-labile linkers (e.g., hydrazone linkers), or reducible linkers (e.g., disulfide linkers). Non-cleavable linkers are attached to the amino acid residues of an antibody through a non-reducible bond, usually known as the thioether bond, which accounting for better plasma stability. Preferably, the linker is stable in the serum and could be cleaved or degraded to release the therapeutic agent in the cell, especially in the lysosome of target cells. In a preferred embodiment, the linker is enzyme-cleavable and could provide covalent bonds between the therapeutic drug and the antibody or antigen-binding fragment of the present disclosure.

In one embodiment, the therapeutic agent is a cytotoxin, wherein the cytotoxin is selected from the group consisting of vinblastines, colchicines, taxanes, auristatins, maytansinoids, calicheamicin, doxorubicin, duocarmycin, SN-38, cryptophycin analogue, deruxtecan, duocarmazine, centanamycin, dolastatins, and pyrrolobenzodiazepine (PBD).

In a specific embodiment, the cytotoxin is an auristatin, such as MMAE, MMAF, MMAD and the like. The synthesis and structure of austenitic compounds are described in US20060229253A1. In a particular embodiment, the therapeutic agent is MMAF or Mertansine (DM1).

In another aspect, the disclosure provides novel linkers and their use in the manufacture of ADCs. For example, in certain embodiments, the linker in an ADC of the present disclosure is selected from LU102, LU104 and LU104′, which have the following structures:

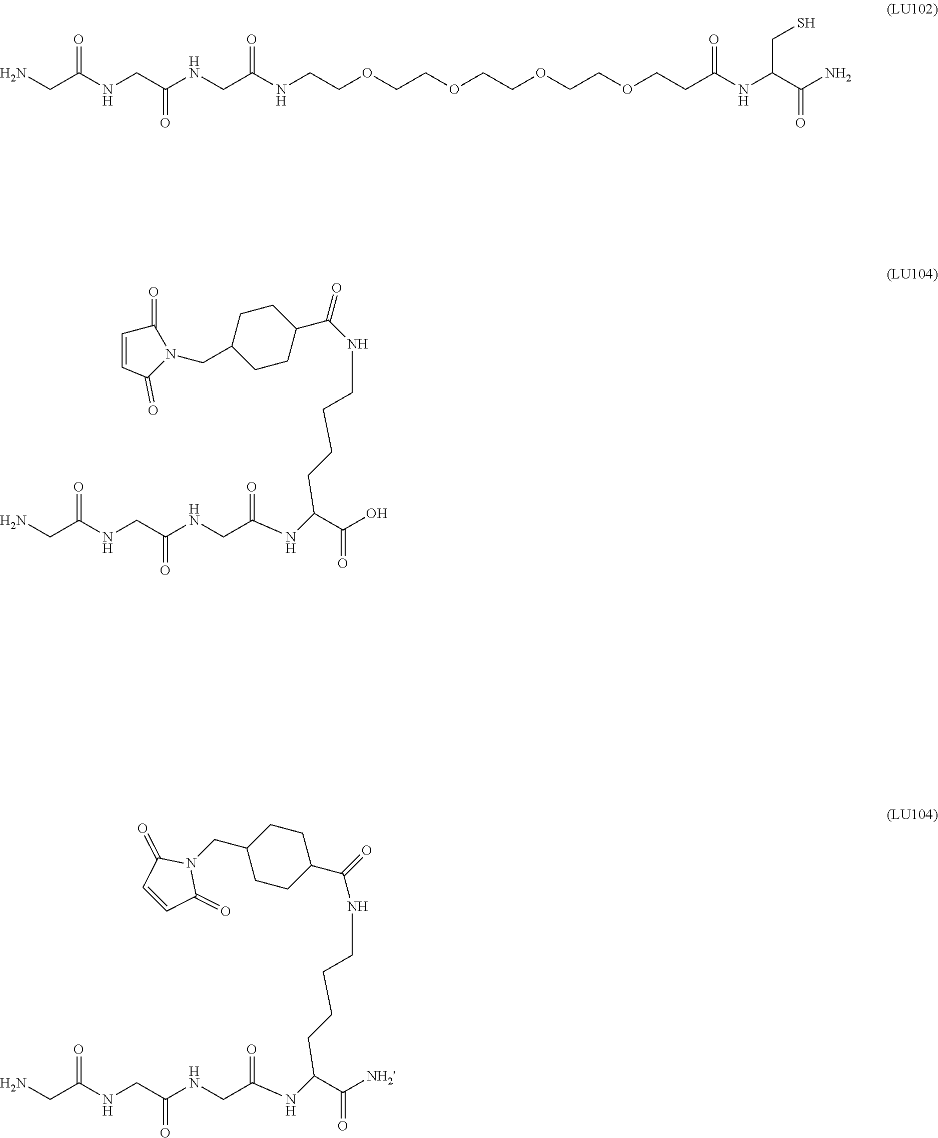

In a particular embodiment, the disclosure provides an antibody-drug conjugate (ADC), comprising an antibody and a therapeutic agent conjugated through a linker, wherein the linker is selected from LU102, LU104 and LU104′.

In an embodiment, the disclosure provides a method of making an ADC, comprising an antibody and a therapeutic agent conjugated through a linker, comprising conjugating a linker to a therapeutic agent, wherein one of the linker or the therapeutic agent comprises a maleimido moiety and the other has a thiol moiety, via reaction of the thiol moiety with the maleimido moiety (e.g., wherein the therapeutic agent is an aurostatin, e.g., MMAD, MMAE or MMAF, which is N-acylated with a maleimido substituent, e.g., maleimidoalkylacyl, e.g., maleimidocaproyl (mc), for example mc-MMAF, and the linker is LU102; or wherein an ADC wherein the therapeutic agent is DM1 (mertansine) and the linker is LU104 or LU104′), reacting the intermediate thus formed with base to open the maleimide ring, coupling this open-ring intermediate to an antibody using sortase-catalyzed coupling, and recovering the ADC thus formed.

In a particular embodiment, the linker is LU104 or LU104′, which comprises a maleimido moiety, the therapeutic agent has a thiol moiety, and the linker is conjugated to the therapeutic agent via reaction of the thiol moiety with the maleimido moiety to form a thiosuccinimide. The therapeutic agent may comprise a thiol moiety itself (for example, DM1) or derivatized to comprise a thiol moiety. It should be understood that the therapeutic agent with or without derivatization is encompassed in the definition of the payload.

In a particular embodiment, the intermediate formed by conjugating the linker to the therapeutic agent (payload) has the structure of Formula (I-1) or Formula (I-2):

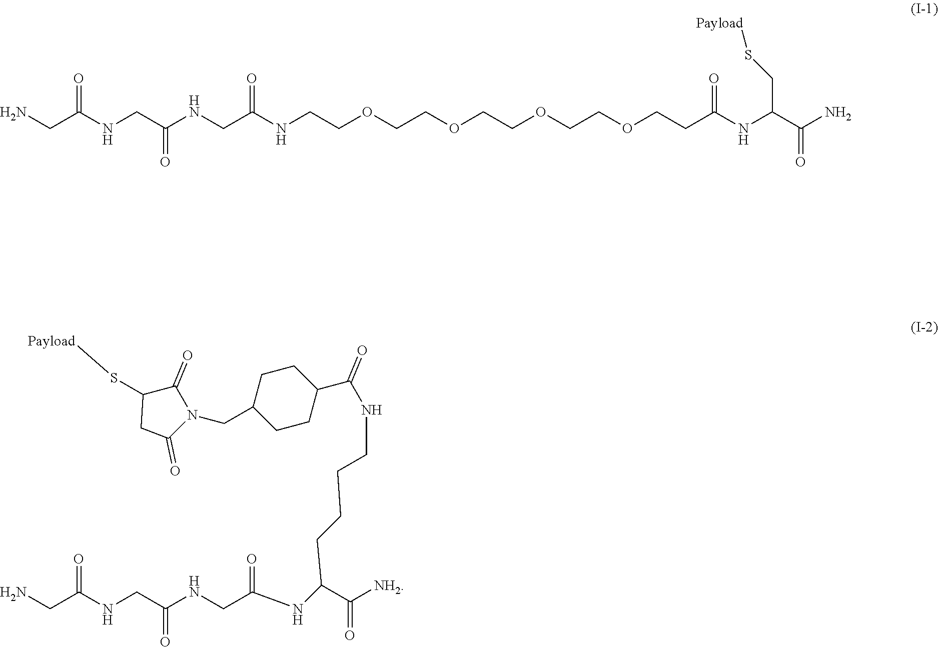

In a particular embodiment, the intermediate as shown in Formula (I-1) is formed by conjugating the linker LU102 to the therapeutic agent. In a particular embodiment, the intermediate as shown in Formula (I-2) is formed by conjugating the linker LU104′ to the therapeutic agent.

In a particular embodiment, the intermediate as shown in Formula (I-2) is reacted with base to open the maleimide ring, and thus forming an open-ring intermediate as shown in Formula (I-2-1) or Formula (I-2-2):

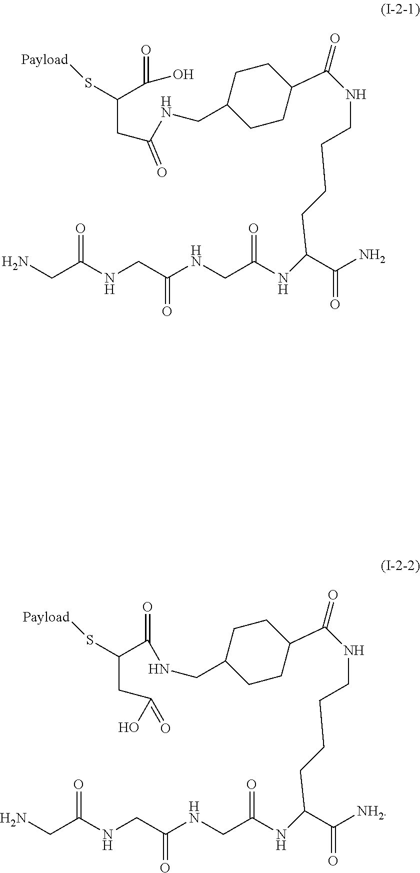

In a particular embodiment, the ADC formed by coupling the intermediate as shown in Formula (I-1) to an antibody has the structure of Formula (II-1):

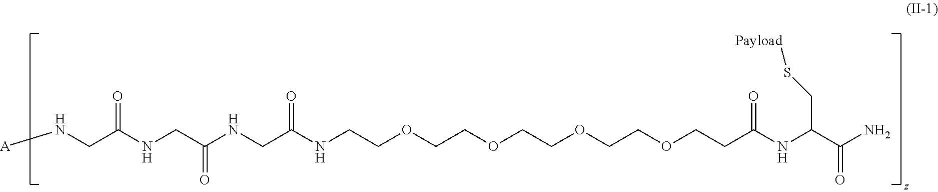

wherein z is an integer of 1 to 20;

A is an antibody.

In a particular embodiment, z is an integer of 1 to 10, for example 1, 2, 3, 4, 5, 6, 7, 8, 9 or 10. In a particular embodiment, z is 2.

In a particular embodiment, the ADC formed by coupling the open-ring intermediate as shown in Formula (I-2-1) to an antibody has the structure of Formula (II-2-1):

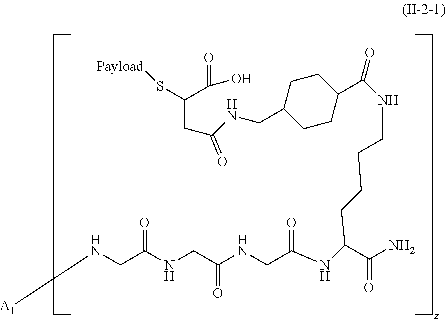

wherein z is as defined above;

A₁ is an antibody.

In a particular embodiment, the ADC formed by coupling open-ring intermediate as shown in Formula (I-2-2) to an antibody has the structure of Formula (II-2-2):

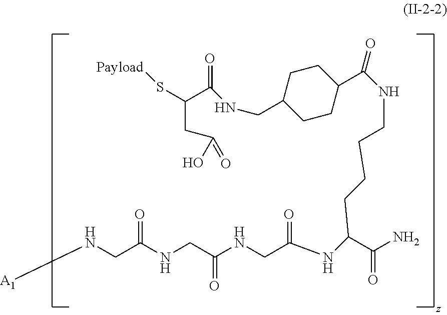

wherein z is as defined above;

A₁ is an antibody.

In a particular embodiment, the payload is mc(ring-open)-MMAF, the ADC of Formula (II-1) has the structure of Formula (1-1) or Formula (1′-1):

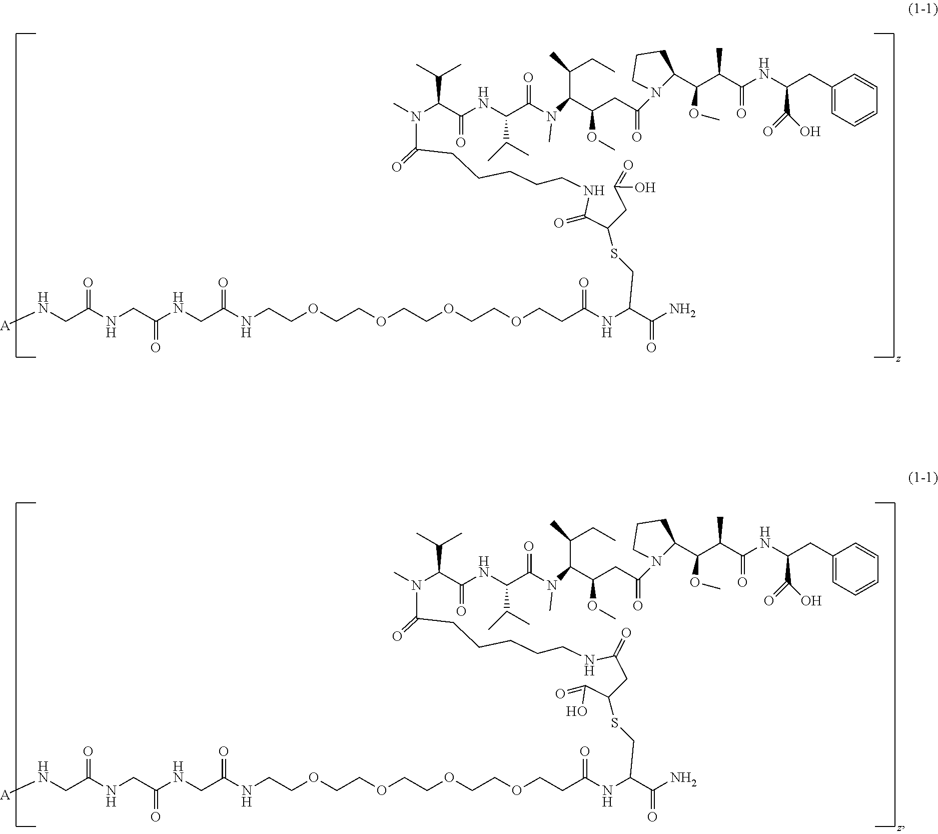

In a particular embodiment, the payload is DM1, the ADC of Formula (II-2-1) has the structure of Formula (2-1):

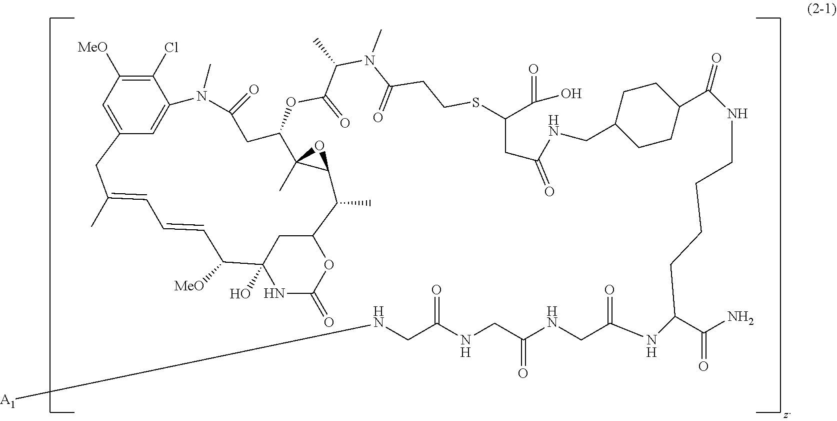

In a particular embodiment, the payload is DM1, the ADC of Formula (II-2-2) has the structure of Formula (2′-1):

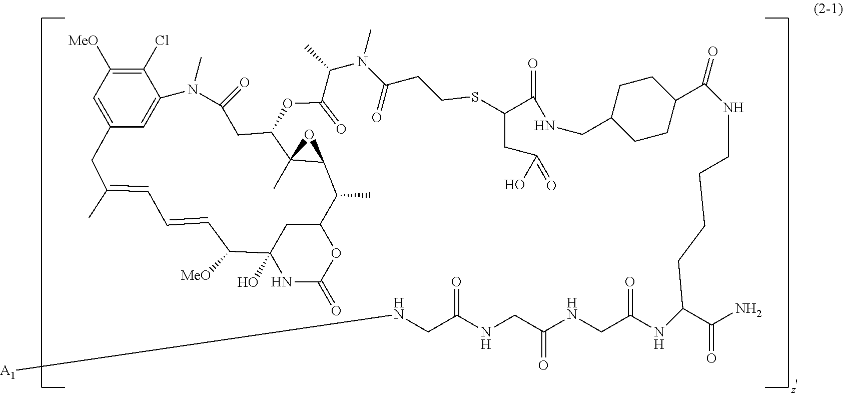

In some embodiments, the therapeutic agent in the ADC is a cytotoxin as described above. In a particular embodiment, the therapeutic agent is an aurostatin, e.g., MMAD, MMAE or MMAF, which is N-acylated with a maleimido substituent, e.g., maleimidoalkylacyl, e.g. maleimidocaproyl (mc), for example mc-MMAF, and the linker is LU102. In a particular embodiment, the therapeutic agent is DM1 (mertansine) and the linker is LU104 or LU104′.

In some embodiments, the antibody in the ADC is an antibody against TROP2. In one embodiment, the antibody in the ADC is the antibody or antigen-binding fragment according to the present disclosure. In a particular embodiment, the antibody in the ADC is Ab0052, Ab0053, Ab0054, Ab0061, Ab0062, Ab0063 or Ab0064, which comprises a light chain and a heavy chain having the amino acid sequences of SEQ ID No. 27 and SEQ ID No. 21 (Ab0052); SEQ ID No. 28 and SEQ ID No. 21 (Ab0053); SEQ ID No. 29 and SEQ ID No. 21 (Ab0054); SEQ ID No. 29 and SEQ ID No. 22 (Ab0061); SEQ ID No. 29 and SEQ ID No. 23 (Ab0062); SEQ ID No. 30 and SEQ ID No. 21 (Ab0063); or SEQ ID No. 30 and SEQ ID No. 22 (Ab0064), respectively (see Table 1). In another embodiment, the antibody in the ADC is Ab0002. Ab002 is a humanized anti-TROP2 antibody (sequence sourced from WHO drug information, INN list 113, Vol. 29, No. 2, 2015) comprising a light chain and a heavy chain having the amino acid sequences of SEQ ID No. 33 and SEQ ID No. 32.

In some embodiments, the antibody or antigen-binding fragment according to the present disclosure is modified to include one or more additional elements prior to preparation of ADCs. Such elements may facilitate the preparation of ADCs, such as a ligase recognition motif (such as a Sortase recognition motif, such as LPETGG) for site-specific conjugation between the antibody and the therapeutic drug under the catalysis of the ligase, or provide spatial separation between the antibody and the therapeutic drug, such as a spacer (such as a spacer sequence containing two amino acids such as GA, and the like). In some embodiments, the ligase is a Sortase, such as Sortase A, Sortase B, Sortase C, Sortase D, Sortase L. plantarum, etc. (US20110321183A1).

In one embodiment, the Sortase is Sortase A from Staphylococcus aureus or Streptococcus pyogenes, the Sortase recognition motif has the amino acid sequence of LPXTGJ, wherein X is any single amino acid that is natural or unnatural; and J is absent, or is an amino acid fragment comprising 1-10 amino acids, optionally labeled. In one embodiment, J is absent. In yet another embodiment, J is an amino acid fragment comprising 1-10 amino acids, wherein each amino acid is independently any natural or unnatural amino acid. In another embodiment, J is Gₘ, wherein m is an integer of 1 to 10. In yet another particular embodiment, the Sortase recognition motif is LPETG or LPETGG. In another embodiment, the Sortase is Sortase B from Staphylococcus aureus, and the Sortase recognition motif is NPQTN. In yet another embodiment, the Sortase is Sortase B from Bacillus anthracis, and the Sortase recognition motif is NPKTG. In another embodiment, the Sortase is Sortase subfamily 5 from Streptomyces coelicolor, and the Sortase recognition motif is LAXTG, wherein X is defined as above. In yet another embodiment, the Sortase is Sortase A from Lactobacillus plantarum, and the Sortase recognition motif is LPQTSEQ. In a particular embodiment, a Sortase recognition motif (LPETGG) and a spacer sequence (GA) (together as GALPETGG, corresponding to SEQ ID No. 25) are introduced at the C-terminus of the light chain of the antibody, wherein the antibody in the ADC is Ab0052, Ab0053, Ab0054, Ab0061, Ab0062, Ab0063, Ab0064 or Ab0002, which comprises a light chain and a heavy chain having the amino acid sequences of SEQ ID No. 17 and SEQ ID No. 21 (Ab0052); SEQ ID No. 18 and SEQ ID No. 21 (Ab0053); SEQ ID No. 19 and SEQ ID No. 21 (Ab0054); SEQ ID No. 19 and SEQ ID No. 22 (Ab0061); SEQ ID No. 19 and SEQ ID No. 23 (Ab0062); SEQ ID No. 20 and SEQ ID No. 21 (Ab0063); SEQ ID No. 20 and SEQ ID No. 22 (Ab0064); or SEQ ID No. 31 and SEQ ID No. 32 (Ab0002), respectively.

In some preferred embodiments, the ADC according to the present disclosure comprises: (i) the antibody or antigen-binding fragment according to the present disclosure, wherein the antibody or antigen-binding fragment is modified to include a ligase recognition motif and/or a spacer sequence, and (ii) a therapeutic agent, wherein the therapeutic agent is coupled to a linker; and the antibody or antigen-binding fragment is coupled to the therapeutic agent through the ligase recognition motif in a site-specific manner under the catalysis of the ligase.

In a particular embodiment, the ADC according to the present disclosure comprises: (i) the antibody or antigen-binding fragment according to the present disclosure, wherein the antibody or antigen-binding fragment is modified (such as at the N-terminus or C-terminus of the light chain or heavy chain) to include a Sortase recognition motif (LPETGG) and a spacer sequence (GA) (together as GALPETGG, corresponding to SEQ ID No. 25), and (ii) a therapeutic agent selected from MMAF and DM1, wherein the therapeutic agent is coupled to a linker selected from LU102, LU104 and LU104′; and the antibody or antigen-binding fragment is coupled to the therapeutic agent through the Sortase recognition motif in a site-specific manner under the catalysis of the Sortase ligase.

In a specific embodiment, the antibody in the ADC of the present disclosure is modified (such as at the N-terminus or C-terminus of the light chain or heavy chain) to include a Sortase recognition motif and a spacer sequence (such as a peptide having the sequence of GALPETGG, corresponding to SEQ ID No. 25), the therapeutic agent in the ADC of the present disclosure is MMAF, which is derivatized with mc to form mc-MMAF (mc is maleimidocaproyl), the linker is LU102, and the ADC has the structure as shown in the following Formula (1) or Formula (1′) or is a mixture thereof:

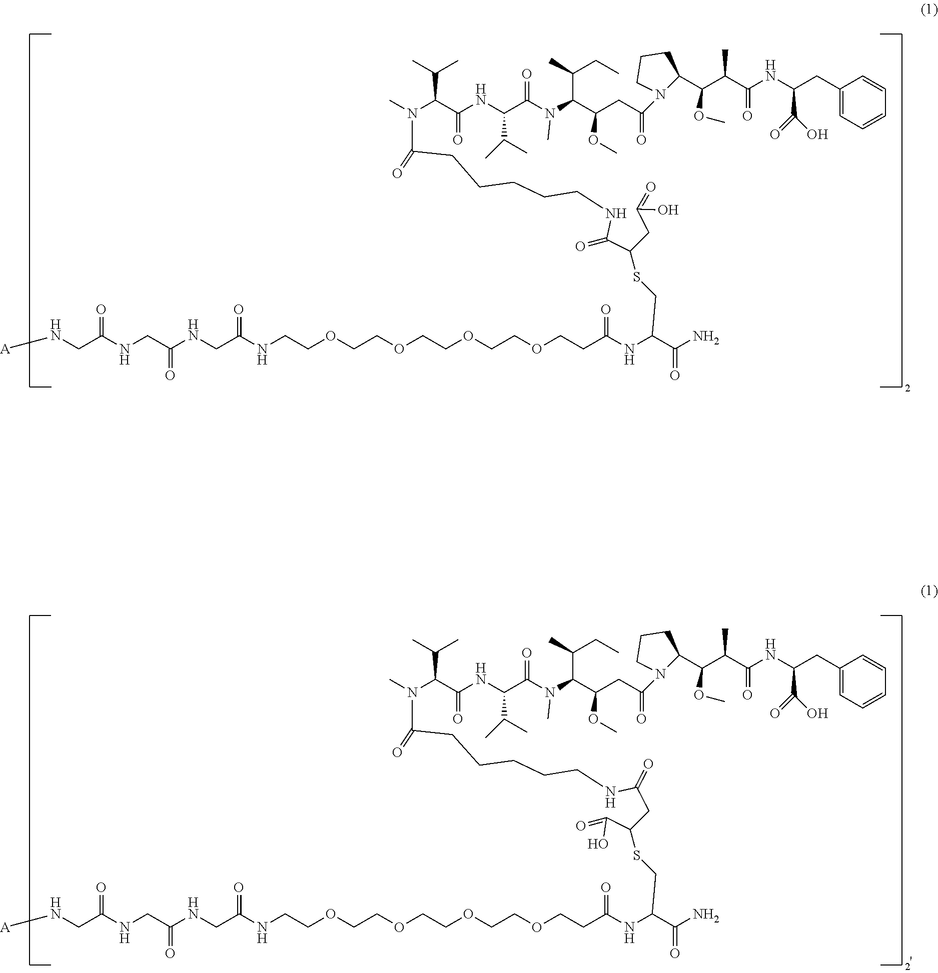

Formulae (1) and (1′) are isomers, wherein A is an antibody, e.g., an antibody to TROP2; preferably, the antibody is selected from Ab0052, Ab0053, Ab0054, Ab0061, Ab0062, Ab0063, Ab0064 and Ab0002. Formula (1) and Formula (1′) fall within the scope of Formula (1-1) and Formula (1′-1), respectively.

In a particular embodiment, in Formula (1) or Formula (1′), the antibody is Ab0052, Ab0053, Ab0054, Ab0061, Ab0062, Ab0063 or Ab0064, which comprises a light chain and a heavy chain having the amino acid sequences of SEQ ID No. 17 and SEQ ID No. 21 (Ab0052); SEQ ID No. 18 and SEQ ID No. 21 (Ab0053); SEQ ID No. 19 and SEQ ID No. 21 (Ab0054); SEQ ID No. 19 and SEQ ID No. 22 (Ab0061); SEQ ID No. 19 and SEQ ID No. 23 (Ab0062); SEQ ID No. 20 and SEQ ID No. 21 (Ab0063); or SEQ ID No. 20 and SEQ ID No. 22 (Ab0064), respectively; and the ADC is DG402, DG502, DG602, DG702, DG802, DG902 or DG1002, respectively.

In another embodiment, in Formula (1) or Formula (1′), the antibody is Ab0002, which comprises a light chain and a heavy chain having the amino acid sequences of SEQ ID No. 31 and SEQ ID No. 32.

In a specific embodiment, the antibody in the ADC of the present disclosure is modified to include a Sortase recognition motif and a spacer sequence (such as a peptide having the sequence of GALPETGG, corresponding to SEQ ID No. 25), the therapeutic agent in the ADC of the present disclosure is DM1, the linker is LU104 or LU104′, and the ADC has the structure as shown in the following Formula (2) or Formula (2′) or is a mixture thereof:

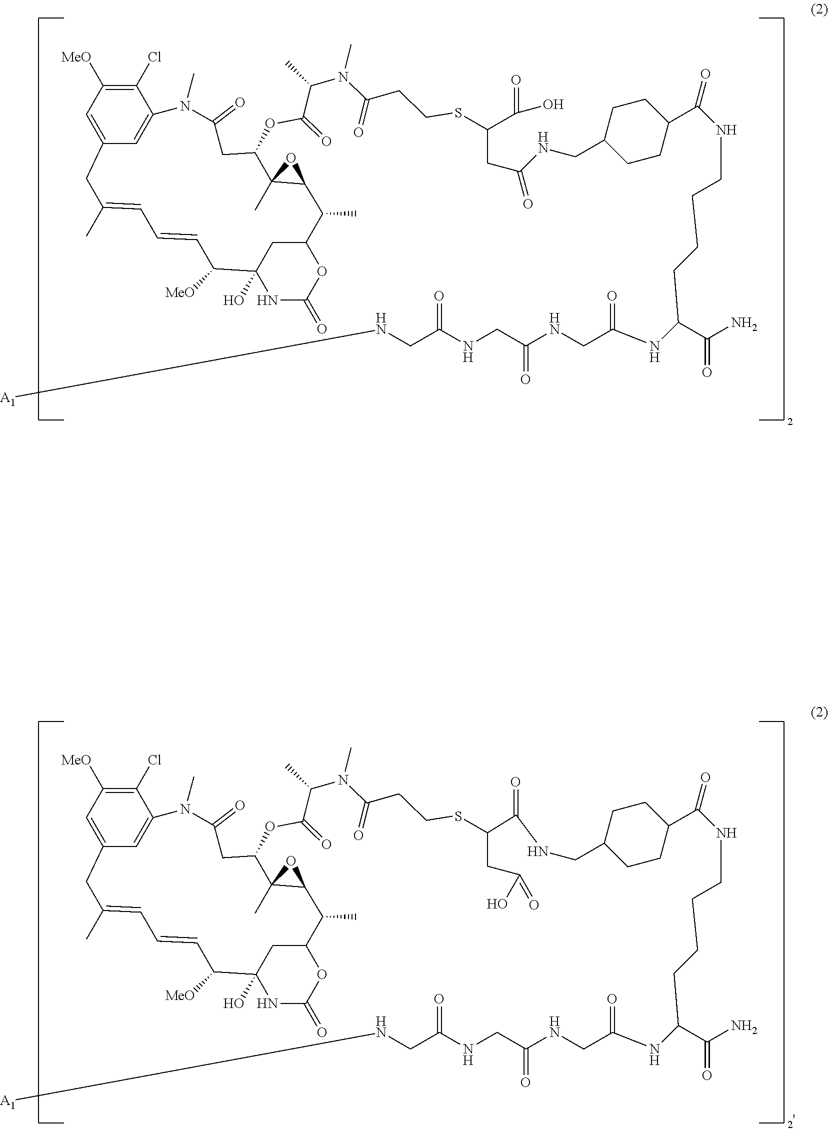

Formulae (2) and (2′) are isomers, wherein A₁ is an antibody, e.g., an antibody to TROP2; preferably, the antibody is selected from Ab0052, Ab0053, Ab0054, Ab0061, Ab0062, Ab0063, Ab0064 and Ab0002. Formula (2) and Formula (2′) fall within the scope of Formula (2-1) and Formula (2′-1), respectively.

In a particular embodiment, in Formula (2) or Formula (2′), the antibody is Ab0052, Ab0053, Ab0054 or Ab0064, which comprises a light chain and a heavy chain having the amino acid sequences of SEQ ID No. 17 and SEQ ID No. 21 (Ab0052); SEQ ID No. 18 and SEQ ID No. 21 (Ab0053); SEQ ID No. 19 and SEQ ID No. 21 (Ab0054); or SEQ ID No. 20 and SEQ ID No. 22 (Ab0064), respectively; and the ADC is DG404, DG504, DG604 or DG1004, respectively.

In another embodiment, in Formula (2) or Formula (2′), the antibody is Ab0002, which comprises a light chain and a heavy chain having the amino acid sequences of SEQ ID No. 31 and SEQ ID No. 32, and the ADC is DG202.

In a specific embodiment, ADCs according to the present disclosure are as shown in Table 2.

TABLE 2

| ADC | Antibody | Linker | Payload |
|---|---|---|---|
| DG402 | Ab0052 (SEQ ID Nos 17 & 21) | LU102 | mc(ring- |
| DG502 | Ab0053 (SEQ ID Nos 18 &21) | | open)- |
| DG602 | Ab0054 (SEQ ID Nos 19 & 21) | | MMAF |
| DG702 | Ab0061 (SEQ ID Nos 19 & 22) | | |
| DG802 | Ab0062 (SEQ ID Nos 19 & 23) | | |
| DG902 | Ab0063 (SEQ ID Nos 20 & 21) | | |
| DG1002 | Ab0064 (SEQ ID Nos 20 & 22) | | |
| DG404 | Ab0052 (SEQ ID Nos 17 & 21) | LU104 or | DM1 |
| DG504 | Ab0053 (SEQ ID Nos 18 &21) | LU104’ | |
| DG604 | Ab0054 (SEQ ID Nos 19 & 21) | (ring-open) | |
| DG1004 | Ab0064 (SEQ ID Nos 20 & 22) | | |
| DG202 | Ab0002 (SEQ ID Nos 31 & 32) | LU104 or | DM1 |
| | | LU104’ | |
| | | (ring-open) | |

In some preferred embodiments, ADCs of the present disclosure are able to target TROP2-positive cells and thereby trace or kill the target cells. In some more preferred embodiments, the antibody-drug conjugate of the present disclosure selectively kill TROP2-positive cancer cells. In some embodiments, said cancer cells include gastric cancer cells, breast cancer cells, urothelial cancer cells, lung cancer cells, liver cancer cells, endometrial cancer cells, head and neck cancer cells, and ovarian cancer cells.

ADCs of the present disclosure can be prepared using desired therapeutic agents and linkers as described above and the antibody or antigen-binding fragment of the present disclosure. Those of skill in the art could choose suitable linkers, therapeutic agents and preparation procedures according to the therapeutic purpose.

### Chimeric Antigen Receptor (CAR) and CAR-Expressing Immune Cells

Also provided are chimeric antigen receptors (CARs) targeting TROP2 and immune cells expressing said CARs. A CAR as described herein is an engineered antigen receptor, comprising:

(i) an extracellular domain comprising at least one antigen-binding fragment of the present disclosure,

(ii) a transmembrane domain, and

(iii) one or more intracellular stimulatory domains of immune effector molecules.

In some embodiments, said at least one antigen-binding fragment comprises a light chain variable region and a heavy chain variable region of the antibody of the present disclosure, preferably, said light chain variable region and heavy chain variable region are the same as an exemplary antibody. In some preferable embodiments, said at least one antigen-binding fragment is a single-chain variable fragment (scFv) comprising at least one antigen-binding fragment of the present disclosure, more preferably, said scFv comprises the same light chain variable region and heavy chain variable region as an exemplary antibody of the present disclosure.

In some embodiments, the transmembrane domain of said CARs could be derived from CD28 or CD8α. In some embodiments, the one or more intracellular stimulatory domains of said CARs comprise a signaling domain, such as the immunoreceptor tyrosine-based activation motif (ITAM) derived from the intracellular tails of CD3 molecules (e.g., CD3ξ), and optionally a co-stimulatory signaling domain, which could be derived from CD7, CD27, CD28, 4-1BB (CD137), CD40, OX40 (CD134), and ICOS etc. In some embodiments, said CARs could further comprise a hinge and spacer region connecting the extracellular domain to the transmembrane domain. In a most preferred embodiment, a CAR as described above possesses both the antigen-binding specificity towards TROP2-positive cells and the effector function of immune effector molecules.

Also provided are nucleic acids encoding any of said CARs and the CAR-expressing immune cells, such as T cells. An immune cell as described above will be directed to TROP2-positive diseased cells (e.g., TROP2-positive cancer cells) and activated to initiate the effector functions, for example, causing the cell death of TROP2-positive cancer cells.

### Pharmaceutical Composition and Treatment

Also provided is a pharmaceutical composition comprising the antibody or antigen-binding fragment thereof of the present disclosure, the therapeutic nucleic acid or expression vector encoding the same, the antibody-drug conjugate of the present disclosure, and/or the immune cell expressing a TROP2-targeting CAR of the present disclosure.

The pharmaceutical composition provided herein can be in various dosage forms, e.g., in solid, semi-solid, liquid, powder, aqueous, or lyophilized forms. Depending on different dosage forms, the pharmaceutical composition could further comprise pharmaceutical acceptable excipients (see, generally, Alfonso R. Gennaro (2000) Remington: The Science and Practice of Pharmacy, 20th Edition. Baltimore, Md.: Lippincott Williams & Wilkins).

Excipients used in solid dosage forms in the preparation of pharmaceutical compositions include, but are not limited to: diluents (e.g., lactose, microcrystalline cellulose and dextrose), binders and adhesives (e.g., acacia, gelatin, starch paste and carboxymethyl cellulose), lubricants (e.g., polyethylene glycol, calcium stearate and steric acid), disintegrants (e.g., starches, cellulose and cross linker polymers), preservatives, vehicles, glidants (e.g., corn starch), sweeteners (e.g., mannitol and saccharin), coating materials (e.g., povidone, ethyl cellulose and synthetic polymers), and plasticizers (e.g., castor oil, diacetylated monoglycerides, and polyethylene glycol). Excipients used in semi-solid dosage forms in the preparation of pharmaceutical compositions include, but are not limited to: structure forming excipients (e.g., cetostearyl alcohol and mineral oils), preservatives (e.g., benzyl alcohol, propylparaben and sodium benzoate), antioxidants (e.g., butyl hydroxyl toluene and butyl hydroxyl anisole), solubilizers (e.g., lanolin and cholesterol), gelling agents (e.g., carboxyl methyl cellulose, hydroxyl propyl cellulose and xanthan gum), and emollients (e.g., glycerin, mineral oil, petrolatum and isopropyl palmitate). Excipients used in liquid dosage forms in the preparation of pharmaceutical compositions include, but are not limited to: solvents (e.g., water, alcohol, acetic acid and syrups), buffers (e.g., phosphate buffers and acetate buffers), antimicrobial preservatives (e.g., benzyl alcohol, butylparaben, phenol and thiomersal), antioxidants (e.g., ascorbic acid, sodium bisulfate, thiourea and butyl hydroxy toluene), chelating agents (e.g., disodium EDTA, dihydroxy ethyl glycine and citric acid), and emulsifying agents (e.g., sodium lauryl sulfate, cetrimide and macrogol esters).

The pharmaceutical composition provided herein can be administered into a subject by any method known in the art, for example, by systemic or local administration. Routes of administration include, but are not limited to, parenteral (e.g., intradermal, intramuscular, intraperitoneal, intravenous, subcutaneous, or intracavity), topical, epidural, or mucosal (e.g. intranasal or oral). The exact dosage to be administered will depend on various factors, such as the therapeutic objectives, the route of administration, and the condition of the subject, for example, the patient's health, body weight, sex, diet, etc. Accordingly, it will be necessary for the therapist to titer the dosage of the pharmaceutical composition and modify the route of administration as required to obtain the optimal therapeutic effect. Generally, the dosage ranges for the administration of the pharmaceutical composition provided herein are those large enough to produce desired effects, in which the TROP2-expressing cells are eliminated.

In a preferred embodiment, the pharmaceutical composition of the present disclosure could be prepared from the anti-TROP2 antibody or antigen-binding fragment of the present disclosure and administered into a human subject in a therapeutically effective amount. A therapeutic dose of the antibodies could be, for example, between preferably 0.1-25 mg/kg body weight per single therapeutic dose and most preferably between 0.1-10 mg/kg body weight for single therapeutic dose. In a particular embodiment, any antibody or antigen-binding fragment of the present disclosure could be formulated in accordance with conventional practice for administration by any suitable route and could generally be in a liquid form (e.g., a solution of the antibody in a sterile physiologically acceptable buffer) for administration by, for example, an intravenous, intraperitoneal, subcutaneous, or intramuscular route. In some embodiments, the pharmaceutical compositions of the present disclosure could be prepared from immune cells (e.g., T cells) expressing a TROP2-targeting CAR of the present disclosure and administered into a subject in a therapeutically effective amount.

The present disclosure also relates to the use of the antibody or antigen-binding fragment thereof of the present disclosure, the nucleic acid or expression vector of the present disclosure, the antibody-drug conjugate of the present disclosure, or the immune cell expressing a TROP2-targeting CAR of the present disclosure, or the pharmaceutical composition of the present disclosure for the manufacture of a medicament for treating disease characterized by TROP2-positive diseased cells.

Further provided is a method of treating a subject with a disease characterized by TROP2-positive diseased cells. Said method comprises administering to the subject in need thereof a therapeutically effective amount of the antibody or antigen-binding fragment thereof of the present disclosure, the nucleic acid or expression vector of the present disclosure, the antibody-drug conjugate of the present disclosure, or the immune cell expressing a TROP2-targeting CAR of the present disclosure, or the pharmaceutical composition of the present disclosure.

Also related is the antibody or antigen-binding fragment thereof of the present disclosure, the nucleic acid or expression vector of the present disclosure, the antibody-drug conjugate of the present disclosure, or the immune cell expressing a TROP2-targeting CAR of the present disclosure, or the pharmaceutical composition of the present disclosure for use in treating disease characterized by TROP2-positive diseased cells.

In some embodiments, said disease is cancer, preferably, TROP2-positive cancer, such as gastric cancer, breast cancer, urothelial cancer, lung cancer, liver cancer, endometrial cancer, head and neck cancer, ovarian cancer or the like. In some preferred embodiments, said subject is a human.

Although the present disclosure has been described with reference to particular embodiments, it is to be understood that these embodiments are merely illustrative of the application and principles of the present disclosure. Modifications can be made without departing from the spirit and scope of the present disclosure.

Specific embodiments according to the present disclosures are listed below:

Embodiment 1. An anti-TROP2 antibody or antigen-binding fragment thereof, wherein the antibody comprises a light chain variable region (V_{L}) and a heavy chain variable region (V_{H}), wherein

the V_{L} comprises:

(i) CDR-L1 having an amino acid sequence of SEQ ID No. 1,

(ii) CDR-L2 having an amino acid sequence of SEQ ID No. 2 or SEQ ID No. 3, and

(iii) CDR-L3 having an amino acid sequence of SEQ ID No. 4;

wherein the V_{H} comprises:

(i) CDR-H1 having an amino acid sequence of SEQ ID No. 5,

(ii) CDR-H2 having an amino acid sequence of SEQ ID No. 6, SEQ ID No. 7 or SEQ ID No. 8, and

(iii) CDR-H3 having an amino acid sequence of SEQ ID No. 9.

Embodiment 2. The antibody according to Embodiment 1 or antigen-binding fragment thereof, wherein

the V_{L} has an amino acid sequence having at least about 85%, at least about 90%, at least about 95% or 100% sequence identity with SEQ ID No. 10, 11, 12, or 13.

Embodiment 3. The antibody according to Embodiment 1 or Embodiment 2 or antigen-binding fragment thereof, wherein

the V_{H} has an amino acid sequence having at least about 85%, at least about 90%, at least about 95% or 100% sequence identity with SEQ ID No. 14, 15, or 16.

Embodiment 4. The antibody according to any one of Embodiments 1-3 or antigen-binding fragment thereof, wherein the antibody comprises a light chain (LC) with an amino acid sequence having at least about 85%, at least about 90%, at least about 95% or 100% sequence identity with SEQ ID No. 17, 18, 19, or 20.

Embodiment 5. The antibody according to any one of Embodiments 1-4 or antigen-binding fragment thereof, wherein the antibody comprises a heavy chain (HC) with an amino acid sequence having at least about 85%, at least about 90%, at least about 95% or 100% sequence identity with SEQ ID No. 21, 22, or 23.

Embodiment 6. The antibody according to any one of Embodiments 1-5 or antigen-binding fragment thereof, wherein the antibody comprises V_{L} and V_{H} of any of the following combinations:

SEQ ID No. 10 and SEQ ID No. 14,

SEQ ID No. 11 and SEQ ID No. 14,

SEQ ID No. 12 and SEQ ID No. 14,

SEQ ID No. 12 and SEQ ID No. 15,

SEQ ID No. 12 and SEQ ID No. 16,

SEQ ID No. 13 and SEQ ID No. 14,

SEQ ID No. 13 and SEQ ID No. 15,

SEQ ID No. 10 and SEQ ID No. 15,

SEQ ID No. 10 and SEQ ID No. 16,

SEQ ID No. 11 and SEQ ID No. 15,

SEQ ID No. 11 and SEQ ID No. 16, or

SEQ ID No. 13 and SEQ ID No. 16.

Embodiment 7. The antibody according to any one of Embodiments 1-6 or antigen-binding fragment thereof, wherein the antibody comprises LC and HC of any of the following combinations:

SEQ ID No. 17 and SEQ ID No. 21,

SEQ ID No. 18 and SEQ ID No. 21,

SEQ ID No. 19 and SEQ ID No. 21,

SEQ ID No. 19 and SEQ ID No. 22,

SEQ ID No. 19 and SEQ ID No. 23,

SEQ ID No. 20 and SEQ ID No. 21,

SEQ ID No. 20 and SEQ ID No. 22,

SEQ ID No. 17 and SEQ ID No. 22,

SEQ ID No. 17 and SEQ ID No. 23,

SEQ ID No. 18 and SEQ ID No. 22,

SEQ ID No. 18 and SEQ ID No. 23, or

SEQ ID No. 20 and SEQ ID No. 23.

Embodiment 8. The antibody according to any one of Embodiments 1-7 or antigen-binding fragment thereof, wherein the antibody or antigen-binding fragment thereof specifically binds to TROP2.

Embodiment 9. The antibody according to Embodiment 8, wherein the TROP2 is a full-length protein or fragment thereof having an epitope to which the antibody according to any one of Embodiments 1-8 binds.

Embodiment 10. The antibody according to Embodiment 8 or Embodiment 9 or antigen-binding fragment thereof, wherein the TROP2 is human or non-human TROP2.

Embodiment 11. The antibody according to any one of Embodiments 1-10 or antigen-binding fragment thereof, wherein the antibody is a humanized antibody.

Embodiment 12. The antibody according to any one of Embodiments 1-11 or antigen-binding fragment thereof, wherein the antibody is an IgG molecule.

Embodiment 13. A nucleic acid, encoding the antibody according to any one of Embodiments 1-12 or antigen-binding fragment thereof.

Embodiment 14. A vector, comprising the nucleic acid molecule according to Embodiment 13.

Embodiment 15. A method of producing the antibody according to any one of Embodiments 1-12, comprising:

(i) transforming a host cell with the nucleic acid according to Embodiment 13 or the vector of Embodiment 14,

(ii) culturing the transformed host cell under conditions suitable for the expression of the nucleic acid molecule or the vector, and

(iii) isolating and purifying the antibody or antigen-binding fragment thereof.

Embodiment 16. An antibody-drug conjugate (ADC), comprising:

(i) the antibody according to any one of Embodiments 1-12 or antigen-binding fragment thereof and

(ii) at least one therapeutic agent.

Embodiment 17. The antibody-drug conjugate according to Embodiment 16, wherein the antibody and the therapeutic agent are conjugated through a linker, which is cleavable or non-cleavable.

Embodiment 18. The antibody-drug conjugate according to Embodiment 17, wherein the linker is a protease-cleavable linker, a pH-labile linker, or a reducible linker.

Embodiment 19. The antibody-drug conjugate according to Embodiment 18, wherein the linker is selected from LU102 and LU104.

Embodiment 20. The antibody-drug conjugate according to any one of Embodiments 16-19, wherein the therapeutic agent is a cytotoxin, preferably mc-MMAF or DM1, for example an antibody-drug conjugate selected from DG1002 and DG 1004.

Embodiment 21. A chimeric antigen receptor comprising:

(i) an extracellular domain comprising the antibody according to any one of Embodiments 1-12 or antigen-binding fragment thereof,

(ii) a transmembrane domain, and

(iii) one or more intracellular stimulatory domains.

Embodiment 22. The chimeric antigen receptor according to Embodiment 21, wherein the antigen-binding fragment is a single-chain Fv fragment (scFv).

Embodiment 23. The chimeric antigen receptor according to Embodiment 21 or Embodiment 22, wherein the transmembrane domain comprises a transmembrane domain derived from CD28 or CD8.

Embodiment 24. The chimeric antigen receptor according to any one of Embodiments 21-23, wherein the intracellular stimulatory domain comprises a signaling domain from CD3ξ.

Embodiment 25. The chimeric antigen receptor according to any one of Embodiments 21-24, wherein the intracellular stimulatory domain is derived from CD7, CD27, CD28, 4-1BB (CD137), CD40, OX40 (CD134), or ICOS.

Embodiment 26. A nucleic acid encoding the chimeric antigen receptor according to any one of Embodiments 21-25.

Embodiment 27. A host cell expressing the chimeric receptor according to any one of Embodiments 21-25.

Embodiment 28. The host cell of Embodiment 27, which is an immune cell, preferably T cell.

Embodiment 29. A pharmaceutical composition comprising:

(i) the antibody according to any one of Embodiments 1-12 or antigen-binding fragment thereof, the nucleic acid according to Embodiment 13, the vector according to Embodiment 14, the antibody-drug conjugate according to any one of Embodiments 16-20, or the host cell according to Embodiment 27 or Embodiment 28; and (ii) a pharmaceutically acceptable excipient.

Embodiment 30. A method of treating a disease in a subject in need thereof, comprising administering to the subject a therapeutically effective amount of the antibody according to any one of Embodiments 1-12 or antigen-binding fragment thereof, the nucleic acid according to Embodiment 13, the vector according to Embodiment 14, the antibody-drug conjugate according to any one of Embodiments 16-20, the host cell according to Embodiment 27 or 28, or the pharmaceutical composition according to Embodiment 29, wherein the disease is TROP2-positive cancer.

Embodiment 31. The method according to Embodiment 30, wherein the TROP2-positive cancer is gastric cancer, breast cancer, urothelial cancer, lung cancer, liver cancer, endometrial cancer, head and neck cancer, or ovarian cancer.

Embodiment 32. The method according to Embodiment 30 comprising administering an antibody-drug conjugate selected from DG1002 and DG1004.

Embodiment 33. An antibody-drug conjugate (ADC), comprising an antibody and a therapeutic agent conjugated through a linker, wherein the linker is selected from LU102 and LU104.

Embodiment 34. The ADC of Embodiment 33 wherein the therapeutic agent is an aurostatin, e.g., MMAD, MMAE or MMAF, which is N-acylated with a maleimido substituent, e.g., maleimidoalkylacyl, e.g. maleimidocaproyl (mc), for example mc-MMAF, and the linker is LU102.

Embodiment 35. The ADC of Embodiment 33 wherein the therapeutic agent is DM1 (mertansine) and the linker is LU104.

Embodiment 36. The ADC of Embodiment 34 having the structure of Formula (1) or Formula (1′) or is a mixture thereof:

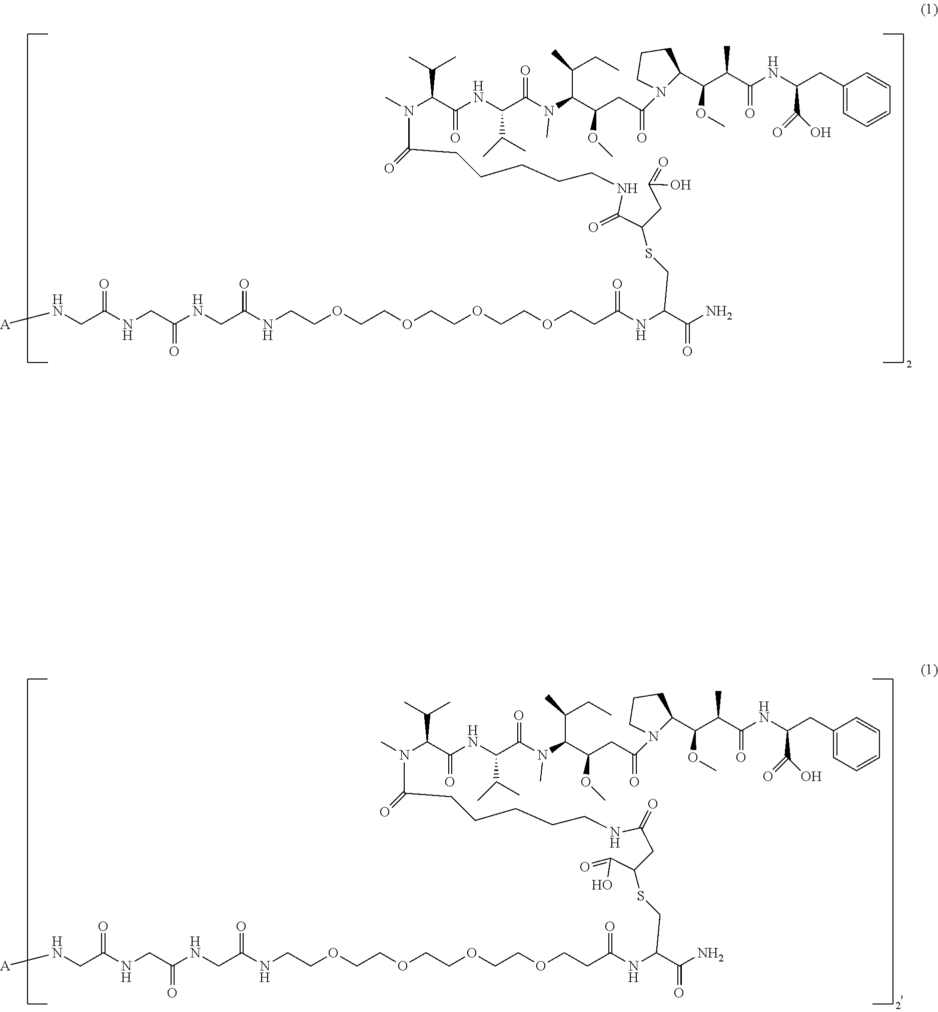

wherein A is an antibody, e.g., an antibody to TROP2, e.g. Ab0052, Ab0053, Ab0054, Ab0061, Ab0062, Ab0063 or Ab0064.

Embodiment 37. The ADC of Embodiment 35 wherein the ADC has the structure of Formula (2) or Formula (2′) or is a mixture thereof:

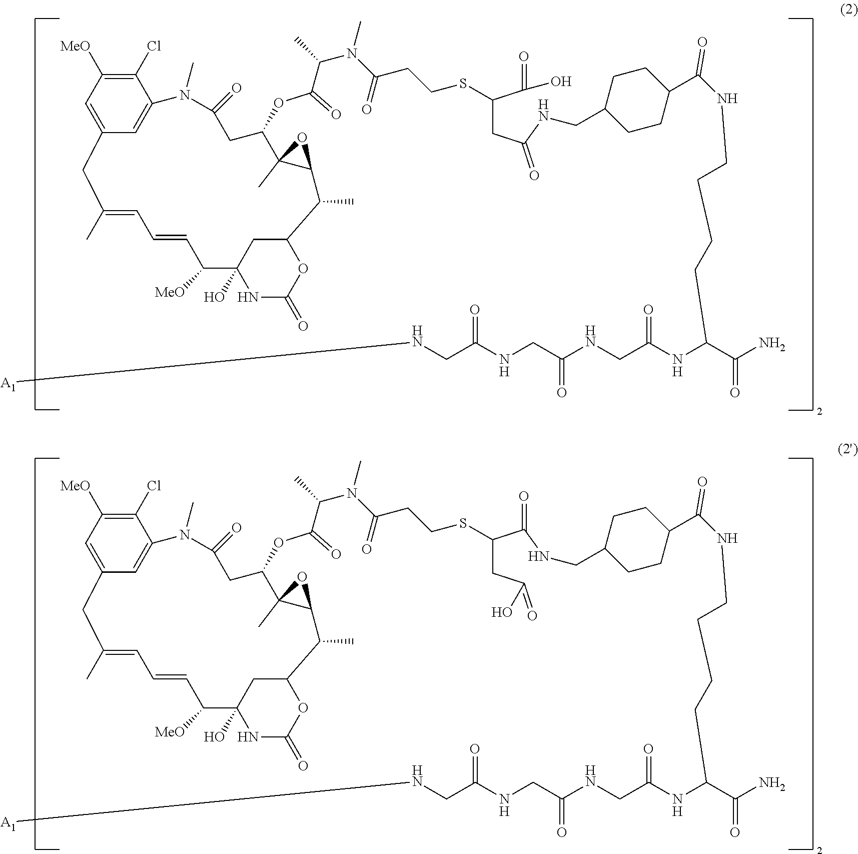

wherein A₁ is an antibody, e.g., an antibody to TROP2, e.g., Ab0052, Ab0053, Ab0054, Ab0061, Ab0062, Ab0063 or Ab0064.

Embodiment 38. The ADC of any of Embodiments 33-37 wherein the antibody is an antibody to TROP2, e.g., Ab0052, Ab0053, Ab0054, Ab0061, Ab0062, Ab0063 or Ab0064.

Embodiment 39. A method of making the antibody-drug conjugate (ADC) of any of Embodiments Embodiment 33-38, comprising

conjugating the linker to the therapeutic agent, wherein one of the linker or the therapeutic agent comprises a maleimido moiety and the other has a thiol moiety, via reaction of the thiol moiety with the maleimido moiety,

reacting the intermediate thus formed with base to open the maleimide ring,

coupling this open-ring intermediate to an antibody using sortase-catalyzed coupling, and

recovering the ADC thus formed.

Embodiment 40. The method of Embodiment 39 wherein

the therapeutic agent is an aurostatin, e.g., MMAD, MMAE or MMAF, which is N-acylated with a maleimido substituent, e.g., wherein the maleimido substituent is maleimidoalkylacyl (e.g. maleimidocaproyl (mc)), for example mc-MMAF;

and the linker is LU102.

Embodiment 41. The method of Embodiment 39 wherein the therapeutic agent is DM1 (mertansine) and the linker is LU104.

Embodiment 42. The compound LU102:

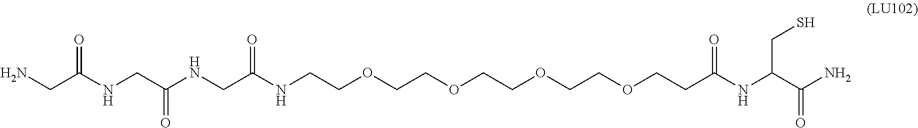 [SMILES: NCC(=O)NCC(=O)NCC(=O)NCCOCCOCCOCCOCCC(=O)NC(CS)C(N)=O]

Embodiment 43. The compound LU104:

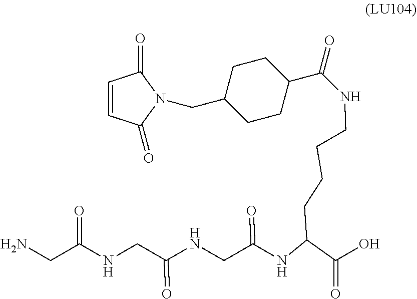 [SMILES: NCC(=O)NCC(=O)NCC(=O)NC(CCCCNC(=O)C1CCC(CN2C(=O)C=CC2=O)CC1)C(=O)O]

Embodiment 44. The use of LU102 or LU104 as a linker in the manufacture of an antibody-drug conjugate.

### Beneficial Effects

An antibody according to the present disclosure has a high affinity to the target TROP2 or fragment thereof with a K_{D} of about 8×10⁻¹⁰ M (0.8 nM), 7×10⁻¹⁰ M (0.7 nM), 6×10⁻¹⁰ M (0.6 nM), 4×10⁻¹⁰ M (0.4 nM), or less. This enables a specific interaction between the target and the antibody.

Further, the antibody according to the present disclosure has good thermal stability. It could be shown as high Tₘ, for example, a Tₘ of 84° C., 85° C., 86° C. or higher, like 84.5° C., 85° C., 85.5° C. Thermal stability is an important biophysical property of biomolecules. High thermal stability confers on the therapeutic antibody longer half-life in unfavorable conditions, such as during the manufacture, shipping, and storage of the antibody. In another aspect, the high thermal stability could enable the antibody according to the present disclosure survive some harsh conditions, for example, chemical reactions during the preparation of conjugated molecules (e.g., ADCs), or unfavorable conditions in the tedious isolation and purification process (e.g., buffers and temperature). Therefore, high thermal stability could greatly facilitate the use of antibodies for many applications.

Moreover, the antibody according to the present disclosure is humanized. Therapeutic antibodies produced in non-human systems could be possibly immunogenic and elicit undesirable and sometimes fatal immune reactions, for example, a murine antibody often induces a human anti-mouse antibody response (HAMA). In addition, immunogenicity can also alter the pharmacokinetics of a therapeutic antibody by impacting clearance, thereby affecting the in vivo efficacy.

In summary, the antibody according to the present disclosure has a high binding affinity to the antigen TROP2 or fragments thereof, good thermal stability and lower immunogenicity. When used as a therapeutic agent, such an antibody or an ADC comprising the antibody shall have at least one of the following: (i) having a longer in vivo half-life, (ii) inducing a lower HAMA response, and (iii) having a higher therapeutic efficacy due to the longer in vivo half-life and lower HAMA response.

### EXAMPLES

### Example 1: Generation of Humanized Anti-Trop2 Antibodies

Methods for constructing humanized antibodies are well known in the art. Briefly, the variable regions of a chimeric antibody are subjected to three-dimensional homology modeling analysis using well-established mouse antibody structures in the protein data bank (PDB). Critical residues within the framework regions that are important for the antigen binding are identified using the same modeling analysis. Then human germline genes with highest sequence similarities to the variable regions of the chimeric antibody are identified and selected as framework donors. Next, the CDRs of the human germline genes are replaced with the CDRs of the chimeric antibody using synthetic oligonucleotides and PCR. To preserve the stability and antigen specificity of the humanized antibody, back-mutations are introduced into the human FR regions, wherein some of the human FR residues are replaced with the counterpart residues in the chimeric antibody.

### 1.1 Homology Modeling of the Variable Regions of Ab0001

The V_{L} and V_{H} regions of Ab0001 (a known chimeric anti-TROP2 antibody) are subjected to three-dimensional homology modeling using the modeling software Bioluminate, using the light chain of the mouse antibody 2D73 (PDB ID: 4BZ2) and heavy chain of the mouse antibody 14G7 (PDB ID: 2Y6S) as templates, respectively. The canonical structures and residues of CDRs are determined. Vernier zone residues in the framework regions are also identified based on the modeling analysis.

### 1.2 Selection of Framework Donors

To select suitable human germline genes as framework (FR) donors (FRs 1-4), the V_{H} and V_{L} sequences of Ab0001 are separately subjected to IgBlast tool in NCBI or IMGT searches against the entire human antibody germline V genes and J genes.

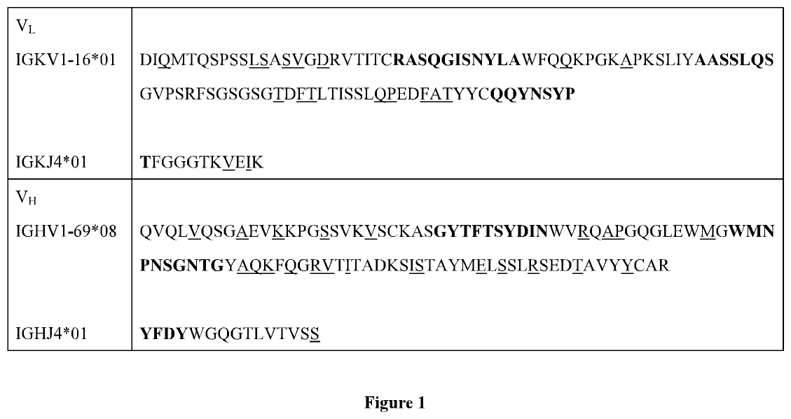

Human heavy chain and light chain germline V genes and J genes with highest sequence similarities to the V_{H} and V_{L} of Ab0001 are selected as framework (FR) donors, respectively. Human heavy chain germline genes IGHV1-69*08 and IGHJ4*01 and light chain germline genes IGKV1-16*01 and IGKJ4*01 are selected as FR donors for the humanization of the V_{H} and V_{L} regions of Ab0001, respectively. The V_{L} and V_{H} sequences of the human germline antibodies are shown in FIG. 1. CDRs are shown in boldface. Framework residues that are different from the counterpart residues in Ab0001 are underlined.

1.3 Sequence Design of the V_{H} and V_{L} Regions of the Humanized Antibodies

The sequences of framework regions of the human germline antibodies and the CDRs of Ab0001 are combined and back-mutations are introduced to generate the sequences of the humanized V_{H} and V_{L} (i.e., the V_{H} and V_{L} sequences of the humanized anti-TROP2 antibody, respectively).

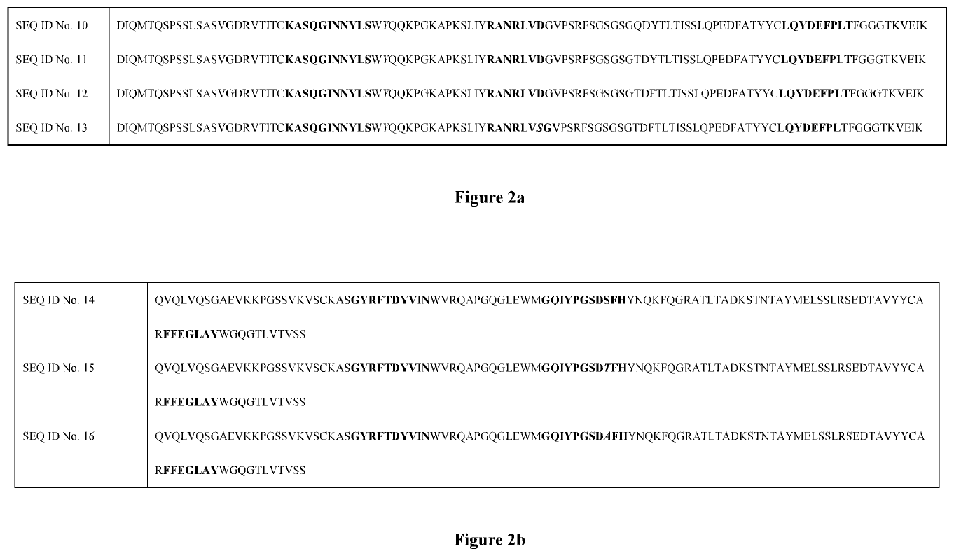

The finalized exemplary sequences of the humanized V_{L} regions (Ab0001-huV3-V_{L}, Ab0001-huV4-V_{L}, Ab0001-huV5-V_{L} and Ab0001-huV8-V_{L}, also SEQ ID No. 10, 11, 12 and 13, respectively) and V_{H} regions (Ab0001-huV4-V_{H}, Ab0001-huV5-V_{H} and Ab0001-huV6-V_{H}, also SEQ ID No. 14, 15 and 16, respectively) are shown in FIGS. 2a and 2b.

For the V_{L} regions, residue VL24-T in CDR-L1 is mutated to K. VL36-F in framework 2 is mutated to Y. Based on the three-dimensional homology modeling in step 1.1, VL69-Q and VL71-Y are Vernier zone residues. VL69-Q and VL71-Y are kept in Ab0001-huV3-V_{L} (SEQ ID No. 10). VL69-Q is mutated to T in Ab0001-huV4-V_{L} (SEQ ID No. 11). VL69-Q is mutated to T and VL71-Y is mutated to F in Ab0001-huV5-V_{L} (SEQ ID No. 12). VL56-D in CDR-L2 is mutated to S in Ab0001-huV8-V_{L} (SEQ ID No. 13) to eliminate the DG motif.

For the V_{H} regions, based on the three-dimensional homology modeling in step 1.1, important framework residues VH68-A and VH70-L (Vernier zone residues), VH61-N (interacts with V_{L} residues), VH77-N (interacts with CDR residues) are kept in Ab0001-huV4-V_{H} (SEQ ID No. 14), Ab0001-huV5-V_{H} (SEQ ID No. 15) and Ab0001-huV6-V_{H} (SEQ ID No. 16). To eliminate the DS motif, VH57-S in CDR-H2 is mutated to T in Ab0001-huV5-V_{H} and mutated to A in Ab0001-huV6-V_{H}.

### 1.4 Construction of Expression Vectors Encoding the Humanized Anti-TROP2 Antibodies

The nucleic acid sequences of IGKV1-16*01, IGKJ4*01 and the nucleic acid sequences encoding the CDRs of light chain of Ab0001 are synthetically assembled to generate the nucleic acid encoding the humanized V_{L}. The nucleic acid sequences of IGHV1-69*08 and IGHJ4*01 and the nucleic acid sequences encoding the CDRs of heavy chain of Ab0001 are synthetically assembled to generate the nucleic acid encoding the humanized V_{H}.

To generate expression vectors encoding the light chains of the humanized anti-TROP2 antibodies, the nucleic acid sequences of the humanized V_{L} obtained above are individually cloned into a pCDNA 3.3 vector (Life technology) containing an N-terminal light chain signal peptide (SEQ ID No. 24), the light chain constant sequence of a human antibody and a sortase recognition motif (LPETGG) and a spacer sequence (GA) (together as GALPETGG, corresponding to SEQ ID No. 25). To generate the expression vectors encoding the heavy chains of the humanized anti-TROP2 antibodies, the nucleic acid sequences of the humanized V_{H} obtained above are individually cloned into a pCDNA 3.3 vector (Life technology) containing an N-terminal heavy chain signal peptide (SEQ ID No. 26) and the heavy chain constant sequence of a human antibody.

### 1.5 Production of the Humanized Anti-TROP2 Antibodies

Plasmids encoding the light and heavy chains of the humanized anti-TROP2 antibodies as prepared in step 1.4 are paired and mixed at a mass ratio of 2:1. Plasmid and PEIMAX (Polyscience) transfection reagent are separately diluted in HEK293F basic medium and then mixed evenly. The mixture is let stand at room temperature and added to the HEK293F seed cell culture. The cell culture is sampled for cell density and viability analysis and supplemented with 10% volume of HEK293F feed medium. Then the culture temperature is shifted to 32° C. for the following culture. At 72 h of incubation, the cell culture is sampled again for cell density and viability analysis. At 144 h of incubation, the cell culture is sampled for cell density and viability analysis.

### 1.6 Purification of the Humanized Anti-TROP2 Antibodies

The antibodies are purified by affinity chromatography following the manufacturer's instruction. Briefly, the chromatography column (BestChrom, Shanghai, China) is packed with the MabSelect SureLX resin (GE Healthcare) and equilibrated with 50 mM Tris, 150 mM NaCl, pH 7.4. Then the supernatant of the cell culture obtained in step 1.5 is applied onto the column. The column is washed with 50 mM Tris, 150 mM NaCl, pH 7.4 to remove non-specifically bound proteins. Then the antibodies are eluted by 50 mM citrate Buffer, pH 3.5 and the antibody-containing eluate is adjusted to pH 6.5 using 1 M Tris-HCl, pH 9.0. Finally, the buffer of the antibodies is exchanged to 50 mM Tris, 150 mM NaCl, pH 7.4 by an Anicon Ultra-15 centrifugal Filter (Merk Millipore). Antibodies of the present disclosure (Ab0052, Ab0053, Ab0054, Ab0061, Ab062, Ab0063, and Ab0064, also see Table 1) are obtained.

### 1.7 Production and Purification of Ab0002

Anti-TROP2 antibody Ab0002 is prepared using a similar method as described in Examples 1.5 and 1.6. The sequence of Ab0002 is obtained from WHO drug information, INN list 113, Vol. 29, No. 2, 2015. Ab002 comprises a heavy chain having the amino acid sequence of SEQ ID No. 32 and a light chain having the amino acid sequence of SEQ ID No. 33. Nucleic acids encoding the light chain and heavy chain of Ab002 are cloned into a pCDNA 3.3 vector. To prepare the ADC comprising Ab0002 according to the present disclosure, the GALPETGG (SEQ ID No. 25) peptide as described above is introduced to the C-terminus of the light chain (SEQ ID No. 33) to give a modified light chain with the amino acid sequence of SEQ ID No. 31.

### Example 2: Binding Kinetics and Affinity Analysis

Binding kinetics and affinity analysis are performed. Surface Plasmon Resonance (SPR) analysis is performed on a Biacore 8K (GE healthcare) with a Sensor Chip Protein A (GE healthcare) following manufacturer's instructions. All measurements are performed at 25° C. in the HBS-EP+ buffer (10 mM HEPES, pH 7.4, 150 mM NaCl, 3 mM EDTA, and 0.05% surfactant P20). About 110-140 RU (Resonance unit) of each of the purified antibodies is captured on flow cells 2, 4, 6, 8, 10, 12, 14, 16 (i.e., the reaction surface) of the sensor chip, respectively. Flow cells 1, 3, 5, 7, 9, 11, 13 and 15 are treated with the HBS-EP+ buffer to serve as reference surfaces.

Serial dilutions of the recombinant extracellular domain of human TROP2 (i.e., the analyte) (Acrobiosystem) (243, 81, 27, 9, 3, 1, 0.333, and 0.111 nM, respectively) are prepared in HBS-EP+ buffer. Capturing of the antibodies is followed by a three-minute injection (association phase) of serial dilutions of TROP2 at a flow rate of 30 μL/min and ten minutes of buffer flow (dissociation phase). The chip surface is regenerated by two pulses of 30-second injection of 10 mM Glycine-HCl pH 1.5 at a flow rate of 50 μL/min. The collected data are processed on a Biacore 8K Evaluation software using methods known in the art, comprising the following steps: (1) setting the response on the y-axis and the start of the injection on the x-axis to zero, (2) performing double referencing by firstly subtracting the reference surface data from the reaction surface data to get the analyte injection curves, and then subtracting the buffer injection curves from the analyte injection curves, and (3) performing the kinetic analysis using the 1:1 binding model with a global fit. The result for each antibody is presented as Ka (on-rate), Kd (off-rate) and K_{D} (affinity constant) (Table 3). As shown in Table 3, all the humanized antibodies and Ab0001 bind to the antigen with similar affinity.

TABLE 3

| Antibody | Ka (1/Ms) | Kd (1/s) | K_{D} (nM) |
|---|---|---|---|
| Ab0001 | 1.09E+06 | 4.41E−04 | 0.41 |
| Ab0052 | 1.03E+06 | 3.21E−04 | 0.31 |
| Ab0053 | 1.01E+06 | 3.26E−04 | 0.32 |
| Ab0054 | 9.75E+05 | 4.16E−04 | 0.43 |
| Ab0061 | 8.74E+05 | 5.45E−04 | 0.62 |
| Ab0062 | 8.89E+05 | 7.19E−04 | 0.81 |
| Ab0063 | 8.62E+05 | 3.94E−04 | 0.46 |
| Ab0064 | 7.22E+05 | 5.47E−04 | 0.76 |

### Example 3: Thermal Stability of the Humanized Antibodies

### 3.1 Tₘ Value Measurement by DSF

A Differential Scanning Fluorimetry (DSF) assay is performed using a LightCycler 480 Real-Time PCR instrument (Roche) following manufacturer's instructions. Briefly, 19 μL of each antibody solution (1 mg/mL) is mixed with 1 μL of 20×SYPRO Orange solution and added to a 96-well plate. The fluorescence emission is collected using a fluorescence resonance energy transfer filter (560-580 nm) with an excitation wavelength of 450-490 nm. During the DSF experiment, the temperature is increased from 25 to 95° C. at an increment of 0.2° C. with an equilibration time of 5 s at each temperature prior to measurement. Data analysis is performed using GraphPad prism. The midpoint of hydrophobic exposure, Tₘ, is defined as the temperature corresponding to the maximum value of the first derivative of the second fluorescence transition.

As show in Table 4, the antibodies of the present disclosure display an unexpected increase in the Tₘ value comparing to Ab0001, i.e., from 79.5° C. to around 85° C., suggesting that the thermal stability of the antibodies of the present disclosure is higher than that of Ab0001.

TABLE 4

| Antibody | Tₘ (° C.) |
|---|---|
| Ab0001 | 79.5 |
| Ab0052 | 85.0 |
| Ab0053 | 85.0 |
| Ab0054 | 85.0 |
| Ab0061 | 85.5 |
| Ab0062 | 84.5 |
| Ab0063 | 84.5 |
| Ab0064 | 85.0 |

### 3.2 Thermal Stability of Antibodies Measured by SEC-HPLC

Heat-induced aggregation and degradation of antibodies is measured by Size Exclusion-High-Performance Liquid Chromatography (SEC-HPLC).

Each antibody is divided into two parts: one part is stored at 2-8° C. to serve as a control, and the other is incubated in a PCR machine at 60° C. for 24 hours (heat-treated). Then, heat-treated or control samples are centrifuged at 12,000 rpm for 5 min, and the supernatants are applied to a SEC-HPLC column to detect the percentages of the monomer (corresponding to the intact antibody), high molecular weight (HMW, corresponding to the antibody aggregates due to aggregation) and low molecular weight (LMW, corresponding to the antibody fragments due to degradation) forms of each antibody. As shown in Table 5, the monomer percentages of all the antibodies are around 98% in the normal storage condition (2-8° C.); after heat treatment (60° C., 24 h), the monomer percentage of Ab0001 decreased to 83.35%, while that of the antibodies of the present disclosure are within a range of 90.47% (Ab0064) to 97.11% (Ab0062), indicating that antibodies of the present disclosure have a much better thermal stability than Ab0001.

TABLE 5

| | | Percentage (%) | | |
|---|---|---|---|---|
| Antibody | Treatment | HMW | Monomer | LMW |
| Ab0001 | 2~8° C. | 0.34 | 99.66 | 0.00 |
| | 60° C., 24 h | 14.64 | 83.35 | 2.00 |
| Ab0052 | 2~8° C. | 1.20 | 98.80 | 0.00 |
| | 60° C., 24 h | 2.29 | 94.78 | 2.93 |
| Ab0053 | 2~8° C. | 1.52 | 98.48 | 0.00 |
| | 60° C., 24 h | 2.47 | 94.67 | 2.86 |
| Ab0054 | 2~8° C. | 0.87 | 99.13 | 0.00 |
| | 60° C., 24 h | 2.04 | 95.22 | 2.74 |
| Ab0061 | 2~8° C. | 0.81 | 99.19 | 0.00 |
| | 60° C., 24 h | 1.78 | 95.50 | 2.72 |
| Ab0062 | 2~8° C. | 0.88 | 99.12 | 0.00 |
| | 60° C., 24 h | 1.70 | 97.11 | 1.19 |
| Ab0063 | 2~8° C. | 0.61 | 99.39 | 0.00 |
| | 60° C., 24 h | 1.78 | 96.38 | 1.84 |
| Ab0064 | 2~8° C. | 2.71 | 97.29 | 0.00 |
| | 60° C., 24 h | 6.09 | 90.47 | 3.44 |

### Example 4: Production of Antibody-Drug Conjugates (ADCs)

The methods for the production of ADCs can be found in e.g. US20170112944A1.

### 4.1 Preparation of Antibodies

A Sortase recognition motif (LPETGG) and a spacer sequence (GA) (together as GALPETGG, corresponding to SEQ ID No. 25) are added to the C-terminus of the light chain of Ab0001 to obtain the modified Ab0001.

### 4.2 Preparation of the Linker

The linker LU102 (see structure shown below) is synthesized by a conventional solid phase polypeptide synthesis using Rink-amide-MBHA-resin or dichloro-resin. Fmoc is used to protect the amino acid and the amino group in the linker. The coupling reagent is selected from HOBT, HOAt/DIC or HATU. After synthesis, the resin is cleaved using trifluoroacetic acid. The product is purified by HPLC, lyophilized and stored for use. Theoretical molecular weight: 538.24, measured: 539.2 [M+H]⁺.

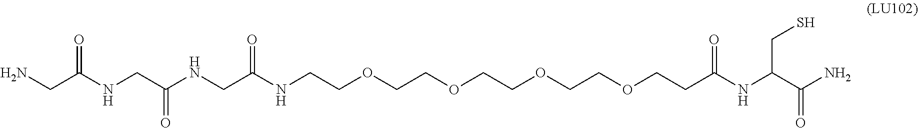 [SMILES: NCC(=O)NCC(=O)NCC(=O)NCCOCCOCCOCCOCCC(=O)NC(CS)C(N)=O]

The linker LU104 is prepared according to the above described method, and its structure is as follows:

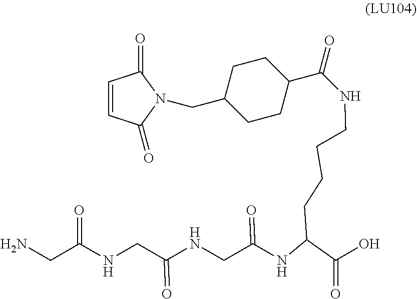 [SMILES: NCC(=O)NCC(=O)NCC(=O)NC(CCCCNC(=O)C1CCC(CN2C(=O)C=CC2=O)CC1)C(=O)O]

As an example, the linker LU104 can be transformed into a C-terminal amidated linker LU104′ by e.g., (1) protecting the terminal NH₂ of glycine by Boc₂O, (2) reacting the product obtained in (1) with NH₃ in the existence of a coupling reagent, which is selected from HOBT, HOAt/DIC and HATU, and (3) deprotecting the terminal NH₂ of glycine. The C-terminal amidated linker LU104′ has the following structure:

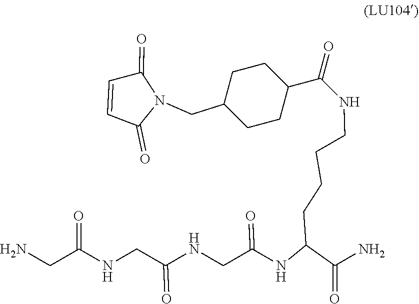 [SMILES: NCC(=O)NCC(=O)NCC(=O)NC(CCCCNC(=O)C1CCC(CN2C(=O)C=CC2=O)CC1)C(N)=O]

Linker LU104 and linker LU104′ can each independently be used to react with the payload to form a linker-payload intermediate.

### 4.3 Preparation of Intermediates

The linker LU102 and mc-MMAF (molar ratio 1.2:1) are weighed and dissolved in water and DMF, respectively, and then thoroughly mixed to give a mixture, which is reacted at 0-40° C. for 0.5-20 h to obtain intermediate IM102 (ring closed). See Formula (i) below. After purification by HPLC, the molecular weight of the intermediate is analyzed by mass spectrometry. Theoretical: 1462.80, measured: 732.41 [M/2+1]⁺, 1463.81 [M+H]⁺.

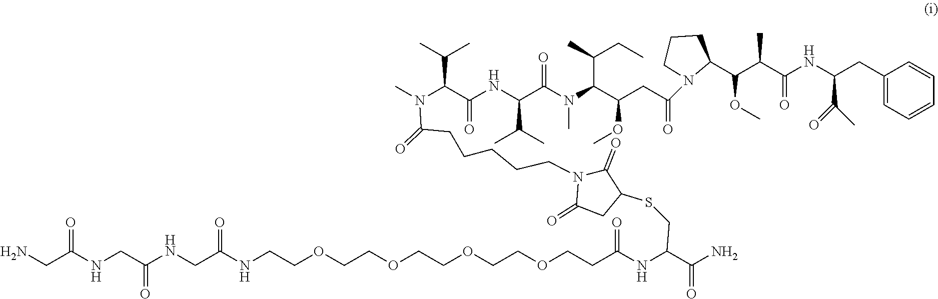

The linker LU104′ and DM1 are covalently linked by the above described method to obtain intermediate IM104 (ring closed) having the structure of Formula (ii):

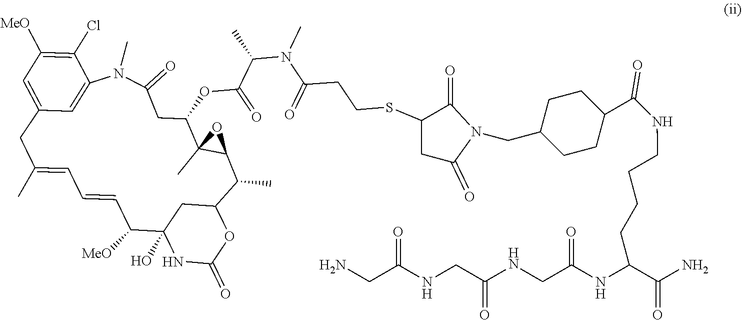 [SMILES: COc1cc2cc(c1Cl)N(C)C(=O)C[C@H](OC(=O)[C@H](C)N(C)C(=O)CCSC1CC(=O)N(CC3CCC(C(=O)NCCCCC(NC(=O)CNC(=O)CNC(=O)CN)C(N)=O)CC3)C1=O)[C@]1(C)O[C@H]1[C@H](C)C1C[C@@](O)(NC(=O)O1)[C@H](OC)/C=C/C=C(\C)C2]

### 4.4 Ring Open of the Intermediate

IM102 (ring closed) is mixed with an appropriate amount of Tris Base solution or other solution that promotes the ring-opening reaction, and the reaction is performed at 0-40° C. for 0.2-20 h. After the reaction is completed, the product is purified by semi-preparative/preparative HPLC to obtain IM102 (ring open), and the structures are as shown in Formula (iii) and Formula (iii′) (shown as isomers). Theoretical molecular weight: 1480.81, measured: 741.41 [M/2+1]⁺, 1481.81 [M+H]⁺.

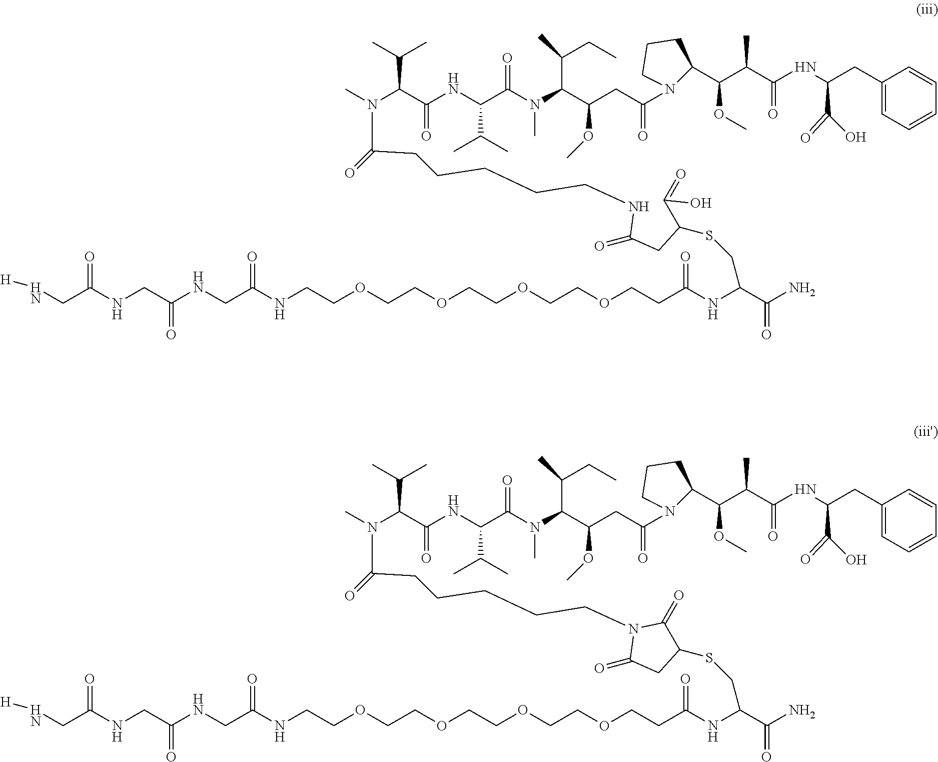 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCNC(=O)CC(SCC(NC(=O)CCOCCOCCOCCOCCNC(=O)CNC(=O)CNC(=O)CN)C(N)=O)C(=O)O)C(C)C ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)CC(SCC(NC(=O)CCOCCOCCOCCOCCNC(=O)CNC(=O)CNC(=O)CN)C(N)=O)C1=O)C(C)C]

The ring opening and purification of the intermediate IM104 (ring closed) is carried out by the above method to obtain the intermediate IM104 (ring open), and the structures are as shown in Formula (iv) and Formula (iv′) (showed as isomers):

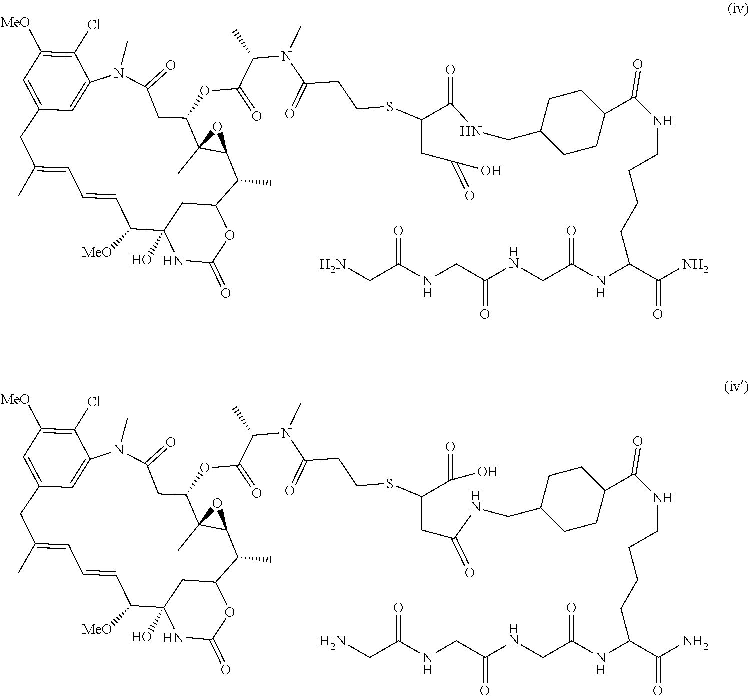 [SMILES: COc1cc2cc(c1Cl)N(C)C(=O)C[C@H](OC(=O)[C@H](C)N(C)C(=O)CCSC(CC(=O)O)C(=O)NCC1CCC(C(=O)NCCCCC(NC(=O)CNC(=O)CNC(=O)CN)C(N)=O)CC1)[C@]1(C)O[C@H]1[C@H](C)C1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCSC(CC(=O)NCC1CCC(C(=O)NCCCCC(NC(=O)CNC(=O)CNC(=O)CN)C(N)=O)CC1)C(=O)O)[C@]1(C)O[C@H]1[C@H](C)C1C[C@@](O)(NC(=O)O1)[C@H](OC)/C=C/C=C(\C)C2]

### 4.5 Preparation of Reference ADCs and ADCs of the Present Disclosure

4.5.1 Preparation of the Reference ADCs (DG002 and DG004) from the Modified Ab0001

IM102 (ring open) is coupled to Ab0001 in a site-specific manner by a ligase (Sortase) to generate DG002 having structures as shown in the above formulae (1) and (1′), wherein Formula (1) and Formula (1′) are isomers. Specific steps are as follows:

### 1) Treatment of Ab0001:

Ab0001 is treated by ultrafiltration, dialysis or desalting column. The storage solution is replaced with a ligase buffer.

### 2) Enzyme-Catalyzed Coupling of DG002:

DG002 is prepared by coupling reaction of Ab0001 with IM102 (ring open), under the catalysis of a wild type Sortase A or a mutant ligase optimized and engineered based thereon. In the ligase buffer, the modified Ab0001 and IM102 (ring open) are thoroughly mixed at a molar ratio of 1:1 to 1:100, and added to a solid phase coupling system. The solid phase coupling system comprised a ligase immobilized on the matrix of the solid phase coupling system. The immobilized ligase catalyzed the coupling reaction of the antibody Ab0001 with IM102 (ring open). The coupling reaction is carried out at 4-40° C. for 0.5-20 h. After the reaction is completed, the reaction mixture is subjected to ultrafiltration or dialysis to remove unreacted intermediate, giving DG002. DG002 is stored at 4° C. or −80° C. in a buffer containing 20 mM citric acid, 200 mM NaCl, pH 5.0.

### 3) SDS-PAGE Detection and Analysis of DG002

The purity and coupling efficiency of DG002 are analyzed by SDS-PAGE. The results are shown in FIG. 1. The coupling reaction occurs at the light chain of the modified Ab0001. The light chain of Ab0001 which is coupled with IM102 (ring open) shows a significant transition in molecular weight compared to that has not undergone a coupling reaction. There is no detectable uncoupled light chain in the coupled product, and the coupling efficiency is as high as 95% or more. The purity of the coupled product meets the expectation.

### 4) HIC-HPLC Detection and Analysis of DG002

The DAR (drug-to-antibody ratio) distribution of DG002 is analyzed by HIC-HPLC. The antibody Ab0001 without cytotoxin is less than 5%; and the coupled product mainly contains DG002 with DAR of 2.

### 5) SEC-HPLC Detection and Analysis of DG002

The degree of high molecular weight aggregation of DG002 is analyzed by SEC-HPLC. The results show that no high molecular weight polymer is detected in DG002, indicating that the coupling reaction conditions are mild and did not cause damage to the antibody structure. The reference ADC DG002 has the structure of Formula (1) or Formula (1′) or is a mixture thereof:

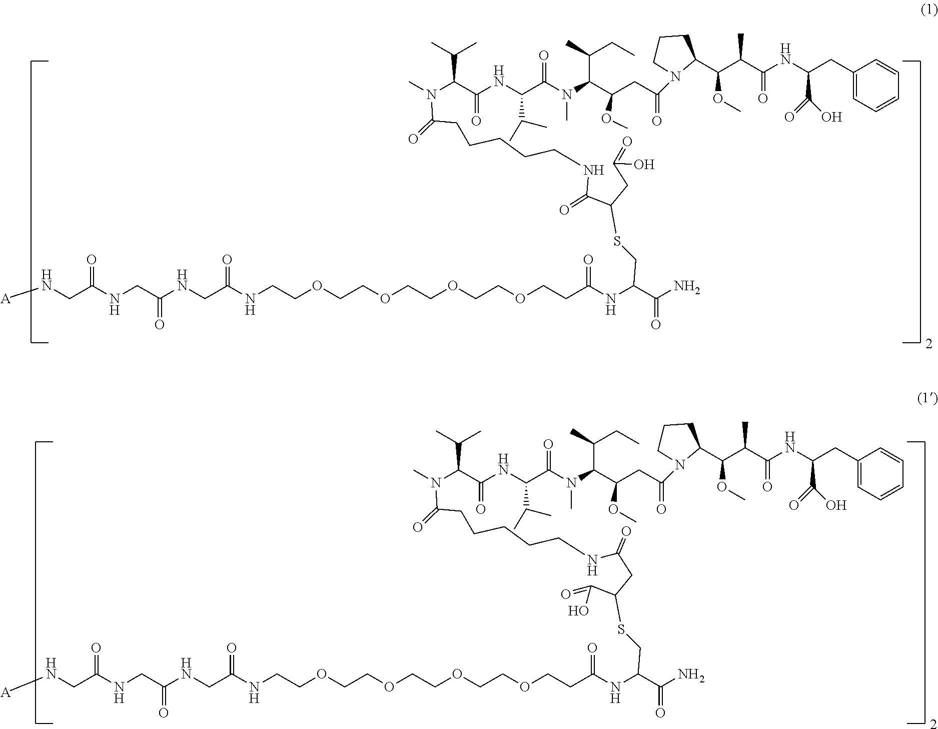

wherein A is Ab0001.

Intermediate IM104 (ring open) is coupled to the modified Ab0001 in a site-specific manner using a method similar to the above point 2) to give DG004, and the structure are as follows (showed as isomers):

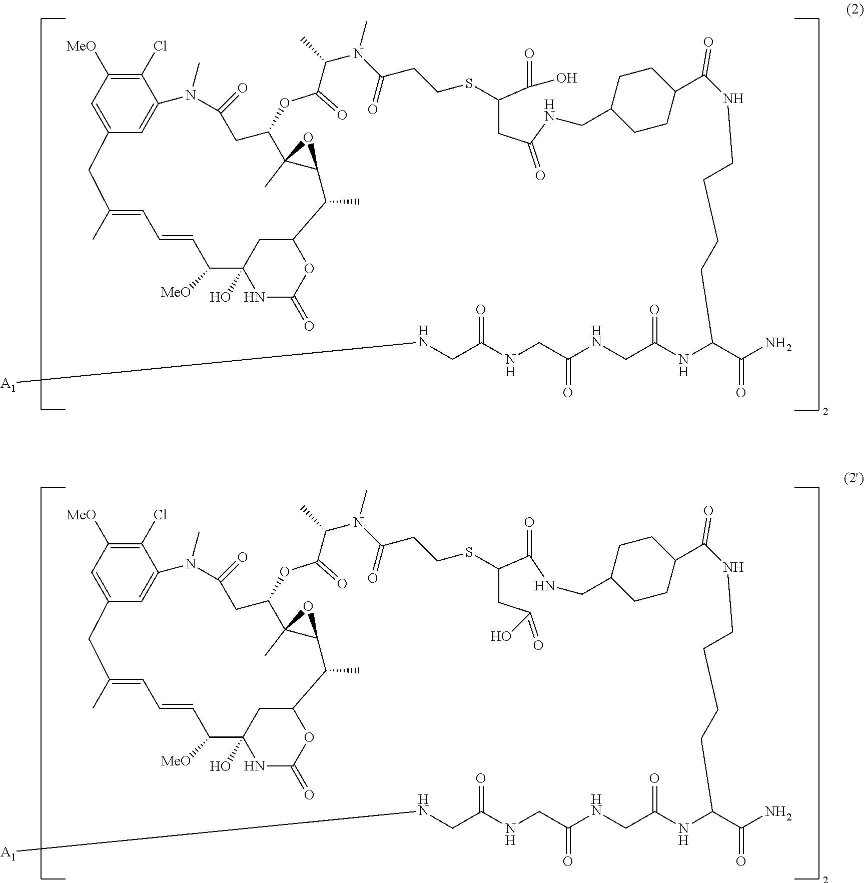

wherein A₁ is Ab0001.

### 4.5.2 Preparation of ADCs of the Present Disclosure

The antibodies of the present disclosure and antibody Ab0002 (a TROP2 antibody, sequence sourced from WHO drug information, INN list 113, Vol. 29, No. 2, 2015) are modified by introducing a spacer sequence (GA) and a Sortase recognition motif (together as GALPETGG, corresponding to SEQ ID No. 25) to the C-terminus of the antibody light chain, and then coupled to the intermediates IM102 (ring open) or IM104 (ring open), using a method similar to 4.5.1 to give the ADCs of the present disclosure. Each of ADCs DG002, DG402, DG502, DG602, DG702, DG802, DG902 and DG 1002 has the structure of Formula (1) or Formula (1′) or is a mixture thereof. Each of ADCs DG004, DG404, DG504, DG604, DG1004 and DG202 has the structure of Formula (2) or Formula (2′) or is a mixture thereof. The ADCs prepared are listed in Table 6.

TABLE 6

| ADC | Antibody | Linker | Payload |
|---|---|---|---|
| DG002 | Ab0001 | LU102 | mc(ring- |
| DG402 | Ab0052 (SEQ ID Nos 17 & 21) | | open)- |
| DG502 | Ab0053 (SEQ ID Nos 18 &21) | | MMAF |
| DG602 | Ab0054 (SEQ ID Nos 19 & 21) | | |
| DG702 | Ab0061 (SEQ ID Nos 19 & 22) | | |
| DG802 | Ab0062 (SEQ ID Nos 19 & 23) | | |
| DG902 | Ab0063 (SEQ ID Nos 20 & 21) | | |
| DG1002 | Ab0064 (SEQ ID Nos 20 & 22) | | |
| DG004 | Ab0001 | LU104’ | DM1 |
| DG404 | Ab0052 (SEQ ID Nos 17 & 21) | (ring-open) | |
| DG504 | Ab0053 (SEQ ID Nos 18 &21) | | |
| DG604 | Ab0054 (SEQ ID Nos 19 & 21) | | |
| DG1004 | Ab0064 (SEQ ID Nos 20 & 22) | | |
| DG202 | Ab0002 ( SEQ ID Nos 31 & 32) | | |

### Example 5 Thermal Stability of the ADCs

### 5.1 Tₘ Value Measurement by DSF

The Tₘ values of ADCs are measured by DSF as described in Example 3. As show in Table 7, the ADCs of the present disclosure displayed an unexpected increase in the Tₘ value comparing to the Ab0001-derived ADCs, i.e., from 77.8° C. (DG002) or 78.1° C. (DG004) to a range of 83.9° C. (DG1004) to 86.9° C. (DG702), suggesting that the thermal stability of the ADCs of the present disclosure is higher than that of the Ab0001-derived ADCs.

TABLE 7

| ADC | Tₘ (° C.) |
|---|---|
| DG002 | 77.8 |
| DG402 | 86.3 |
| DG502 | 86.1 |
| DG602 | 86.5 |
| DG702 | 86.9 |
| DG802 | 86.0 |
| DG902 | 86.3 |
| DG1002 | 86.0 |
| DG004 | 78.1 |
| DG404 | 84.1 |
| DG604 | 84.4 |
| DG1004 | 83.9 |

### 5.2 Thermal Stability Measured by SEC-HPLC

Heat-induced aggregation and degradation of ADCs is measured by SEC-HPLC as described in Example 3. As shown in Table 8, the monomer percentages of all the ADCs are around 98% in the normal storage condition (2-8° C.); after heat treatment (60° C., 24 h), the monomer percentages of DG004 and DG002 (Ab0001-derived ADCs) dramatically decreased to 58.09% and 60.31%, respectively, while that of the ADCs of the present disclosure are within a range of 92.50% (DG402) to 96.07% (DG404), indicating that the ADCs of the present disclosure have a much better thermal stability than the Ab0001-derived ADCs.

TABLE 8

| | | Percentage (%) | | |
|---|---|---|---|---|
| ADC | Treatment | HMW (%) | Monomer (%) | LMW (%) |
| DG004 | 2~8° C. | 2.38 | 97.51 | 0.11 |
| | 60° C., 24 h | 40.09 | 58.09 | 1.82 |
| DG002 | 2~8° C. | 0.91 | 98.21 | 0.88 |
| | 60° C., 24 h | 36.53 | 60.31 | 3.16 |
| DG404 | 2~8° C. | 1.09 | 98.91 | 0.00 |
| | 60° C., 24 h | 3.26 | 96.07 | 0.67 |
| DG402 | 2~8° C. | 1.52 | 98.43 | 0.05 |
| | 60° C., 24 h | 4.66 | 92.50 | 2.83 |
| DG502 | 2~8° C. | 1.49 | 98.51 | 0.00 |
| | 60° C., 24 h | 4.00 | 93.34 | 2.66 |
| DG604 | 2~8° C. | 0.99 | 99.01 | 0.00 |
| | 60° C., 24 h | 3.54 | 95.72 | 0.74 |
| DG602 | 2~8° C. | 1.78 | 98.17 | 0.04 |
| | 60° C., 24 h | 3.69 | 93.72 | 2.59 |
| DG702 | 2~8° C. | 0.88 | 99.12 | 0.00 |
| | 60° C., 24 h | 2.52 | 95.36 | 2.12 |
| DG802 | 2~8° C. | 0.88 | 99.12 | 0.00 |
| | 60° C., 24 h | 2.69 | 95.28 | 2.04 |
| DG902 | 2~8° C. | 0.79 | 99.21 | 0.00 |
| | 60° C., 24 h | 2.87 | 95.54 | 1.59 |
| DG1004 | 2~8° C. | 0.55 | 99.45 | 0.00 |
| | 60° C., 24 h | 3.52 | 95.74 | 0.74 |
| DG1002 | 2~8° C. | 0.74 | 99.26 | 0.00 |
| | 60° C., 24 h | 2.97 | 94.95 | 2.08 |

### Example 6: Inhibitory Activity of ADCs on Tumor Cell Growth

ADCs comprising antibodies are prepared as in Example 4. Tumor cell lines NCI-N87 (ATCC, cat #CRL-5822), MDA-MB-468 (ATCC cat #HTB-132), SK-BR-3 (ATCC, cat #HTB-30), MCF-7 (ATCC, cat #HTB-22), FaDu (ATCC, cat #HTB-43) and COLO 205 (ATCC, cat #CCL-222) are used to assess the cell growth inhibitory activity of the ADCs. TROP2 is highly expressed in NCI-N87, FaDu, MDA-MB-468 and SK-BR-3 and moderately expressed in MCF-7 and COL0205.

Tumor cells in good conditions with confluency of about 80% are digested, seeded in 96-well plates and cultured overnight. After cell adhesion, the ADCs are added at different concentrations (30, 10, 3.333, 1.111, 0.370, 0.123, 0.041, 0.014, 0.005 nM, respectively). After 72 h incubation at 37° C., cell viability under different ADC concentrations is determined using CellTiter-Glo® luminescent kit (Promega) and analyzed using a Prism 6 software to obtain the IC₅₀ value of each ADC (Table 9). As shown in Table 9, the ADCs of the present disclosure effectively inhibited the growth of tumor cells.

TABLE 9

| | IC₅₀ (nM) | | | | | |
|---|---|---|---|---|---|---|
| | NCI- | MDA- | SK- | MCF- | | |
| ADC | N87 | MB-468 | BR-3 | 7 | COLO205 | FaDu |
| DG002 | 0.017 | 0.045 | 0.020 | 0.038 | 0.039 | 0.029 |
| DG402 | 0.027 | ND | ND | ND | ND | ND |
| DG502 | 0.027 | ND | ND | ND | ND | ND |
| DG602 | 0.028 | 0.058 | 0.025 | 0.047 | 0.064 | 0.042 |
| DG702 | 0.021 | ND | ND | ND | ND | ND |
| DG802 | 0.021 | ND | ND | ND | ND | ND |
| DG902 | 0.021 | 0.036 | 0.023 | 0.031 | 0.037 | 0.040 |
| DG1002 | 0.016 | 0.034 | 0.019 | 0.030 | 0.038 | 0.039 |
| DG004 | 0.058 | ND | ND | ND | ND | ND |
| DG404 | 0.082 | ND | ND | ND | ND | ND |
| DG504 | 0.059 | ND | ND | ND | ND | ND |
| DG604 | 0.065 | ND | ND | ND | ND | ND |
| DG1004 | 0.051 | ND | ND | ND | ND | ND |
| DG202 | 0.108 | 0.262 | ND | ND | ND | 0.173 |

ND: Not determined

### Example 7: In Vivo Tumor Inhibition Efficacy of ADCs

Xenograft tumor models are established using BALB/c nude mice and a human gastric carcinoma cell line NCI-N87. NCI-N87 cells are maintained in vitro as a monolayer culture in RPMI 1640 medium (Gibco) supplemented with 10% fetal bovine serum, 100 U/mL penicillin and 100 μg/mL streptomycin, at 37° C. with 5% CO₂. The cells are routinely subcultured twice weekly. NCI-N87 cells growing in an exponential growth phase are harvested and counted for inoculation. BALB/c nude mice, (female, six- to eight-week-old, weighing approximately 18˜22 g) are purchased from Beijing Vital River Laboratory Animal Co., LTD or other certified vendors.

Each mouse is inoculated subcutaneously at the right scapular region with 10×10⁶ NCI-N87 cells in 0.2 mL of PBS. The animals are randomized into eight groups of six. ADCs as shown in Table 10 are intravenously injected into the mice when the average tumor volume reached approximately 100-200 mm³. Groups treated with vehicle (0.9% saline) or the naked Ab0064 antibody (10 mg/kg) served as control.

TABLE 10

| ADC | Antibody | Linker | Payload | Dosage (mg/kg) |
|---|---|---|---|---|
| DG1002 | Ab0064 | LU102 | mc(ring- | 1 |
| | | | open)- | 2 |
| | | | MMAF | 4 |
| DG1004 | | LU104’ | DM1 | 2.5 |
| | | (ring-open) | | 5 |
| | | | | 10 |
| DG202 | Ab0002 | LU104’ | DM1 | 3 |
| | | (ring-open) | | |

Tumor size is measured twice weekly in two dimensions using a caliper and expressed as volume (V) in mm³ using the formula: V=0.5 a×b², wherein a is the length and b is the width of the tumor, respectively.

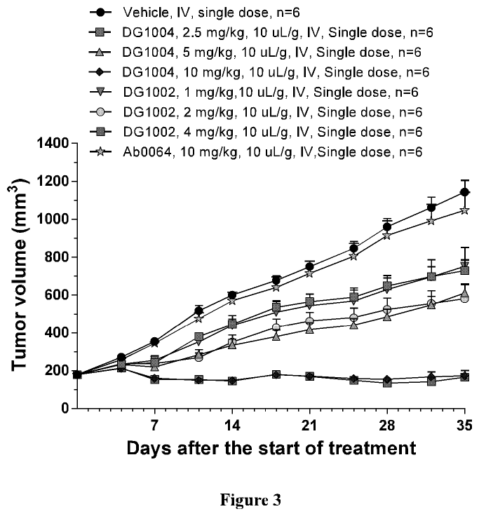

As show in Figure. 3, DG1002 and DG1004 inhibited tumor growth at the dose of 4 mg/kg and 10 mg/kg, respectively. Therefore, the ADCs of the present disclosure are effective in tumor growth inhibition at a lower dosage.

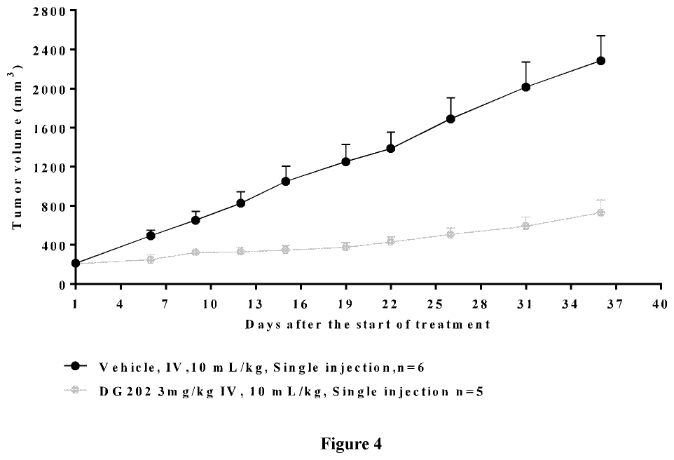

As show in Figure. 4, DG202 inhibited tumor growth at the dose of 3 mg/kg. Therefore, the ADCs of the present disclosure are effective in tumor growth inhibition at a lower dosage.

Sequence Listing

| SEQ | | |
|---|---|---|
| ID No. | Description | SEQUENCE |
| 1 | CDR-L1 | KASQGINNYLS |
| 2 | CDR-L2 | RANRLVD |
| 3 | CDR-L2 | RANRLVS |
| 4 | CDR-L3 | LQYDEFPLT |
| 5 | CDR-H1 | GYRFTDYVIN |
| 6 | CDR-H2 | GQIYPGSDSFH |
| 7 | CDR-H2 | GQIYPGSDTFH |
| 8 | CDR-H2 | GQIYPGSDAFH |
| 9 | CDR-H3 | FFEGLAY |
| 10 | Ab0001-Hu | DIQMTQSPSSLSASVGDRVTITCKASQGINNYLSWYQQK |
| | V3-V_{L} | PGKAPKSLIYRANRLVDGVPSRFSGSGSGQDYTLTISSLQ |
| | | PEDFATYYCLQYDEFPLTFGGGTKVEIK |
| 11 | Ab0001-Hu | DIQMTQSPSSLSASVGDRVTITCKASQGINNYLSWYQQK |
| | V4-V_{L} | PGKAPKSLIYRANRLVDGVPSRFSGSGSGTDYTLTISSLQ |
| | | PEDFATYYCLQYDEFPLTFGGGTKVEIK |
| 12 | Ab0001-Hu | DIQMTQSPSSLSASVGDRVTITCKASQGINNYLSWYQQK |
| | V5-V_{L} | PGKAPKSLIYRANRLVDGVPSRFSGSGSGTDFTLTISSLQP |
| | | EDFATYYCLQYDEFPLTFGGGTKVEIK |
| 13 | Ab0001-Hu | DIQMTQSPSSLSASVGDRVTITCKASQGINNYLSWYQQK |
| | V8-V_{L} | PGKAPKSLIYRANRLVSGVPSRFSGSGSGTDFTLTISSLQP |
| | | EDFATYYCLQYDEFPLTFGGGTKVEIK |
| 14 | Ab0001-Hu | QVQLVQSGAEVKKPGSSVKVSCKASGYRFTDYVINWVR |
| | V4-V_{H} | QAPGQGLEWMGQIYPGSDSFHYNQKFQGRATLTADKST |
| | | NTAYMELSSLRSEDTAVYYCARFFEGLAYWGQGTLVTVS |
| | | s |
| 15 | Ab0001-Hu | QVQLVQSGAEVKKPGSSVKVSCKASGYRFTDYVINWVR |
| | V5-V_{H} | QAPGQGLEWMGQIYPGSDTFHYNQKFQGRATLTADKST |
| | | NTAYMELSSLRSEDTAVYYCARFFEGLAYWGQGTLVTVS |
| | | s |
| 16 | Ab0001-Hu | QVQLVQSGAEVKKPGSSVKVSCKASGYRFTDYVINWVR |
| | V6-V_{H} | QAPGQGLEWMGQIYPGSDAFHYNQKFQGRATLTADKST |
| | | NTAYMELSSLRSEDTAVYYCARFFEGLAYWGQGTLVTVS |
| | | S |
| 17 | Ab0001-Hu | DIQMTQSPSSLSASVGDRVTITCKASQGINNYLSWYQQK |
| | V3-LC | PGKAPKSLIYRANRLVDGVPSRFSGSGSGQDYTLTISSLQ |
| | | PEDFATYYCLQYDEFPLTFGGGTKVEIKRTVAAPSVFIF |
| | | PPSDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGECGALPETGG |
| 18 | Ab0001-Hu | DIQMTQSPSSLSASVGDRVTITCKASQGINNYLSWYQQK |
| | V4-LC | PGKAPKSLIYRANRLVDGVPSRFSGSGSGTDYTLTISSLQ |
| | | PEDFATYYCLQYDEFPLTFGGGTKVEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGECGALPETGG |
| 19 | Ab0001-Hu | DIQMTQSPSSLSASVGDRVTITCKASQGINNYLSWYQQKP |
| | V5-LC | GKAPKSLIYRANRLVDGVPSRFSGSGSGTDFTLTISSLQP |
| | | EDFATYYCLQYDEFPLTFGGGTKVEIKRTVAAPSVFIFP |
| | | PSDEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGECGALPETGG |
| 20 | Ab0001-Hu | DIQMTQSPSSLSASVGDRVTITCKASQGINNYLSWYQQK |
| | V8-LC | PGKAPKSLIYRANRLVSGVPSRFSGSGSGTDFTLTISSLQP |
| | | EDFATYYCLQYDEFPLTFGGGTKVEIKRTVAAPSVFIFPPS |
| | | DEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGECGALPETGG |
| 21 | Ab0001-Hu | QVQLVQSGAEVKKPGSSVKVSCKASGYRFTDYVINWVR |
| | V4-HC | QAPGQGLEWMGQIYPGSDSFHYNQKFQGRATLTADKST |
| | | NTAYMELSSLRSEDTAVYYCARFFEGLAYWGQGTLVTVS |
| | | SASTKGPSVFPLAPSSKSTSGGTAALGCLVKDYFPEPVTV |
| | | SWNSGALTSGVHTFPAVLQSSGLYSLSSWTVPSSSLGTQ |
| | | TYICNVNHKPSNTKVDKKVEPKSCDKTHTCPPCPAPELL |
| | | GGPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSREEMTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 22 | Ab0001-Hu | QVQLVQSGAEVKKPGSSVKVSCKASGYRFTDYVINWVR |
| | V5-HC | QAPGQGLEWMGQIYPGSDTFHYNQKFQGRATLTADKST |
| | | NTAYMELSSLRSEDTAVYYCARFFEGLAYWGQGTLVTVS |
| | | SASTKGPSVFPLAPSSKSTSGGTAALGCLVKDYFPEPVTV |
| | | SWNSGALTSGVHTFPAVLQSSGLYSLSSWTVPSSSLGTQ |
| | | TYICNVNHKPSNTKVDKKVEPKSCDKTHTCPPCPAPELL |
| | | GGPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSREEMTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 23 | Ab0001-Hu | QVQLVQSGAEVKKPGSSVKVSCKASGYRFTDYVINWVR |
| | V6-HC | QAPGQGLEWMGQIYPGSDAFHYNQKFQGRATLTADKST |
| | | NTAYMELSSLRSEDTAVYYCARFFEGLAYWGQGTLVTVS |
| | | SASTKGPSVFPLAPSSKSTSGGTAALGCLVKDYFPEPVTV |
| | | SWNSGALTSGVHTFPAVLQSSGLYSLSSWTVPSSSLGTQ |
| | | TYICNVNHKPSNTKVDKKVEPKSCDKTHTCPPCPAPELL |
| | | GGPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSREEMTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 24 | N-terminal | MDMRVPAQLLGLLLLWLRGARC |
| | light chain | |
| | signal | |
| | peptide | |
| 25 | Sortase | GALPETGG |
| | recognition | |
| | motif and | |
| | spacer | |
| | sequence | |
| 26 | N-terminal | MEFGLSWVFLVAILKGVQC |
| | heavy chain | |
| | signal | |
| | peptide | |
| 27 | Ab0001-Hu | DIQMTQSPSSLSASVGDRVTITCKASQGINNYLSWYQQK |
| | V3-LC | PGKAPKSLIYRANRLVDGVPSRFSGSGSGQDYTLTISSLQ |
| | | PEDFATYYCLQYDEFPLTFGGGTKVEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| 28 | Ab0001-Hu | DIQMTQSPSSLSASVGDRVTITCKASQGINNYLSWYQQK |
| | V4-LC | PGKAPKSLIYRANRLVDGVPSRFSGSGSGTDYTLTISSLQ |
| | | PEDFATYYCLQYDEFPLTFGGGTKVEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| 29 | Ab0001-Hu | DIQMTQSPSSLSASVGDRVTITCKASQGINNYLSWYQQK |
| | V5-LC | PGKAPKSLIYRANRLVDGVPSRFSGSGSGTDFTLTISSLQP |
| | | EDFATYYCLQYDEFPLTFGGGTKVEIKRTVAAPSVFIFPPS |
| | | DEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| 30 | Ab0001-Hu | DIQMTQSPSSLSASVGDRVTITCKASQGINNYLSWYQQK |
| | V8-LC | PGKAPKSLIYRANRLVSGVPSRFSGSGSGTDFTLTISSLQP |
| | | EDFATYYCLQYDEFPLTFGGGTKVEIKRTVAAPSVFIFPPS |
| | | DEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| 31 | Ab0002-LC | DIQLTQSPSSLSASVGDRVSITCKASQDVSIAVAWYQQKP |
| | | GKAPKLLIYSASYRYTGVPDRFSGSGSGTDFTLTISSLQPE |
| | | DFAVYYCQQHYITPLTFGAGTKVEIKRTVAAPSVFIFPPSD |
| | | EQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGECGALPETGG |
| 32 | Ab0002-HC | QVQLQQSGSELKKPGASVKVSCKASGYTFTNYGMNWV |
| | | KQAPGQGLKWMGWINTYTGEPTYTDDFKGRFAFSLDTS |
| | | VSTAYLQISSLKADDTAVYFCARGGFGSSYWYFDVWGQ |
| | | GSLVTVSSASTKGPSVFPLAPSSKSTSGGTAALGCLVKD |
| | | YFPEPVTVSWNSGALTSGVHTFPAVLQSSGLYSLSSVVT |
| | | VPSSSLGTQTYICNVNHKPSNTKVDKRVEPKSCDKTHTC |
| | | PPCPAPELLGGPSVFLFPPKPKDTLMISRTPEVTCVVVDV |
| | | SHEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPGK |
| 33 | Ab0002-LC | DIQLTQSPSSLSASVGDRVSITCKASQDVSIAVAWYQQKP |
| | | GKAPKLLIYSASYRYTGVPDRFSGSGSGTDFTLTISSLQPE |
| | | DFAVYYCQQHYITPLTFGAGTKVEIKRTVAAPSVFIFPPSD |
| | | EQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |

1. An anti-TROP2 antibody or antigen-binding fragment thereof, wherein the antibody comprises a light chain variable region (V_{L}) and a heavy chain variable region (V_{H}), wherein
the V_{L} comprises:
(i) CDR-L1 having an amino acid sequence of SEQ ID No. 1,
(ii) CDR-L2 having an amino acid sequence of SEQ ID No. 2 or SEQ ID No. 3, and
(iii) CDR-L3 having an amino acid sequence of SEQ ID No. 4;
wherein the V_{H} comprises:
(i) CDR-H1 having an amino acid sequence of SEQ ID No. 5,
(ii) CDR-H2 having an amino acid sequence of SEQ ID No. 6, SEQ ID No. 7 or SEQ ID No. 8, and
(iii) CDR-H3 having an amino acid sequence of SEQ ID No. 9.

2. The antibody according to claim 1 or antigen-binding fragment thereof, wherein
the V_{L} has an amino acid sequence having at least about 85%, at least about 90%, at least about 95% or 100% sequence identity with SEQ ID No. 10, 11, 12, or 13.

3. The antibody according to claim 1 or antigen-binding fragment thereof, wherein
the V_{H} has an amino acid sequence having at least about 85%, at least about 90%, at least about 95% or 100% sequence identity with SEQ ID No. 14, 15, or 16.

4. The antibody according to claim 1 or antigen-binding fragment thereof, wherein the antibody comprises a light chain (LC) with an amino acid sequence having at least about 85%, at least about 90%, at least about 95% or 100% sequence identity with SEQ ID No. 17 or 27, 18 or 28, 19 or 29, or 20 or 30.

5. The antibody according to claim 1 or antigen-binding fragment thereof, wherein the antibody comprises a heavy chain (HC) with an amino acid sequence having at least about 85%, at least about 90%, at least about 95% or 100% sequence identity with SEQ ID No. 21, 22, or 23.

6. The antibody according to claim 1 or antigen-binding fragment thereof, wherein the antibody comprises V_{L} and V_{H} of any of the following combinations:
SEQ ID No. 10 and SEQ ID No. 14,
SEQ ID No. 11 and SEQ ID No. 14,
SEQ ID No. 12 and SEQ ID No. 14,
SEQ ID No. 12 and SEQ ID No. 15,
SEQ ID No. 12 and SEQ ID No. 16,
SEQ ID No. 13 and SEQ ID No. 14,
SEQ ID No. 13 and SEQ ID No. 15,
SEQ ID No. 10 and SEQ ID No. 15,
SEQ ID No. 10 and SEQ ID No. 16,
SEQ ID No. 11 and SEQ ID No. 15,
SEQ ID No. 11 and SEQ ID No. 16, or
SEQ ID No. 13 and SEQ ID No. 16.

7. The antibody according to claim 1 or antigen-binding fragment thereof, wherein the antibody comprises LC and HC of any of the following combinations:
SEQ ID No. 17 or 27 and SEQ ID No. 21,
SEQ ID No. 18 or 28 and SEQ ID No. 21,
SEQ ID No. 19 or 29 and SEQ ID No. 21,
SEQ ID No. 19 or 29 and SEQ ID No. 22,
SEQ ID No. 19 or 29 and SEQ ID No. 23,
SEQ ID No. 20 or 30 and SEQ ID No. 21,
SEQ ID No. 20 or 30 and SEQ ID No. 22,
SEQ ID No. 17 or 27 and SEQ ID No. 22,
SEQ ID No. 17 or 27 and SEQ ID No. 23,
SEQ ID No. 18 or 28 and SEQ ID No. 22,
SEQ ID No. 18 or 28 and SEQ ID No. 23, or
SEQ ID No. 20 or 30 and SEQ ID No. 23.

8. The antibody according to claim 1 or antigen-binding fragment thereof, wherein the antibody or antigen-binding fragment thereof specifically binds to TROP2, optionally wherein the TROP2 is a full-length protein or a fragment thereof having an epitope to which the antibody according to claim 1 binds, and further optionally wherein the TROP2 is human or non-human TROP2.

9. (canceled)

10. (canceled)

11. The antibody according to claim 1 or antigen-binding fragment thereof, wherein the antibody is a humanized antibody, optionally wherein the antibody is an IgG molecule.

12. (canceled)

13. A nucleic acid, encoding the antibody according to claim 1 or antigen-binding fragment thereof.

14. A vector, comprising the nucleic acid molecule according to claim 13.

15. A method of producing the antibody according to claim 1, comprising:
(i) transforming a host cell with a nucleic acid encoding the antibody,
(ii) culturing the transformed host cell under conditions suitable for the expression of the nucleic acid molecule or the vector, and optionally
(iii) isolating and purifying the antibody or antigen-binding fragment thereof.

16. An antibody-drug conjugate (ADC), comprising:
(i) the antibody according to claim 1 or antigen-binding fragment thereof; and
(ii) at least one therapeutic agent.

17. The ADC according to claim 16, wherein the antibody and the therapeutic agent are conjugated through a linker, which is cleavable or non-cleavable, optionally wherein the linker is a protease-cleavable linker, a pH-labile linker, or a reducible linker.

18. (canceled)

19. The ADC according to claim 17, wherein the linker is selected from LU102, LU104 and LU104′, which have the following structures:

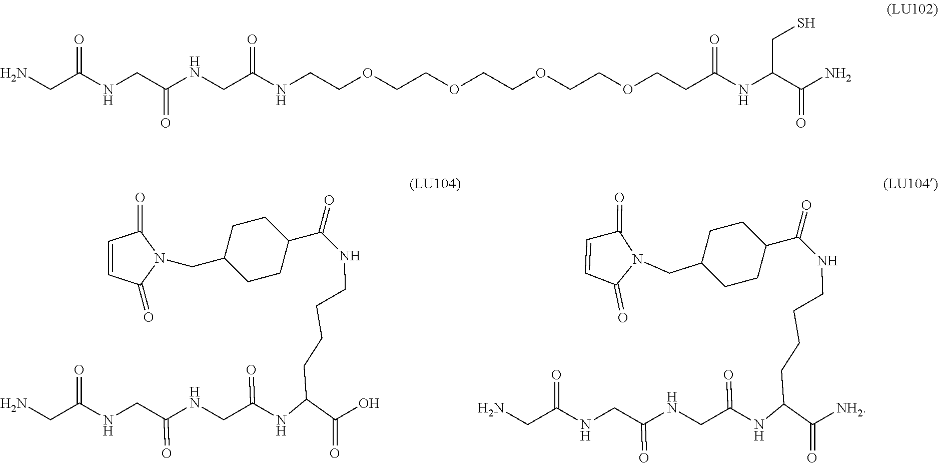 [SMILES: NCC(=O)NCC(=O)NCC(=O)NCCOCCOCCOCCOCCC(=O)NC(CS)C(N)=O ; NCC(=O)NCC(=O)NCC(=O)NC(CCCCNC(=O)C1CCC(CN2C(=O)C=CC2=O)CC1)C(=O)O ; NCC(=O)NCC(=O)NCC(=O)NC(CCCCNC(=O)C1CCC(CN2C(=O)C=CC2=O)CC1)C(N)=O]

20. The ADC according to claim 19, wherein the ADC has the structure of Formula (II-1), Formula (II-2-1) or Formula (II-2-2), or is a mixture of Formula (II-2-1) and Formula (II-2-2): 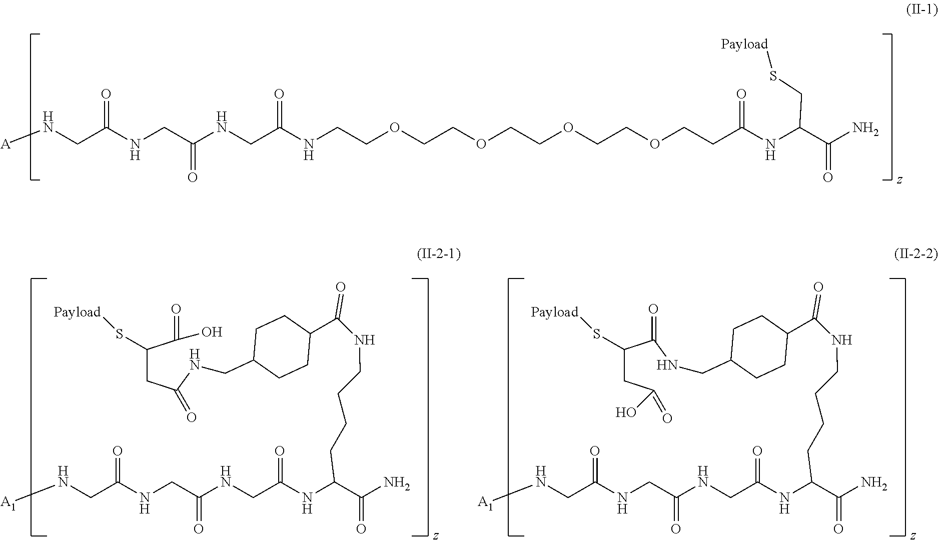
wherein z is an integer of 1 to 20;
wherein Payload is a therapeutic agent;
wherein A and A₁ are each independently selected from the antibody according to any one of claims 1-12 or antigen-binding fragment thereof; preferably, A and A₁ are each independently selected from Ab0052, Ab0053, Ab0054, Ab0061, Ab0062, Ab0063 and Ab0064;
preferably, z is 2.

21. The ADC according to claim 16, wherein the therapeutic agent is a cytotoxin, preferably mc-MMAF or DM1, for example an antibody-drug conjugate selected from DG1002 and DG 1004.

22. The ADC according to claim 21, wherein the ADC has the structure of Formula (1-1) or Formula (1′-1) or is a mixture thereof: 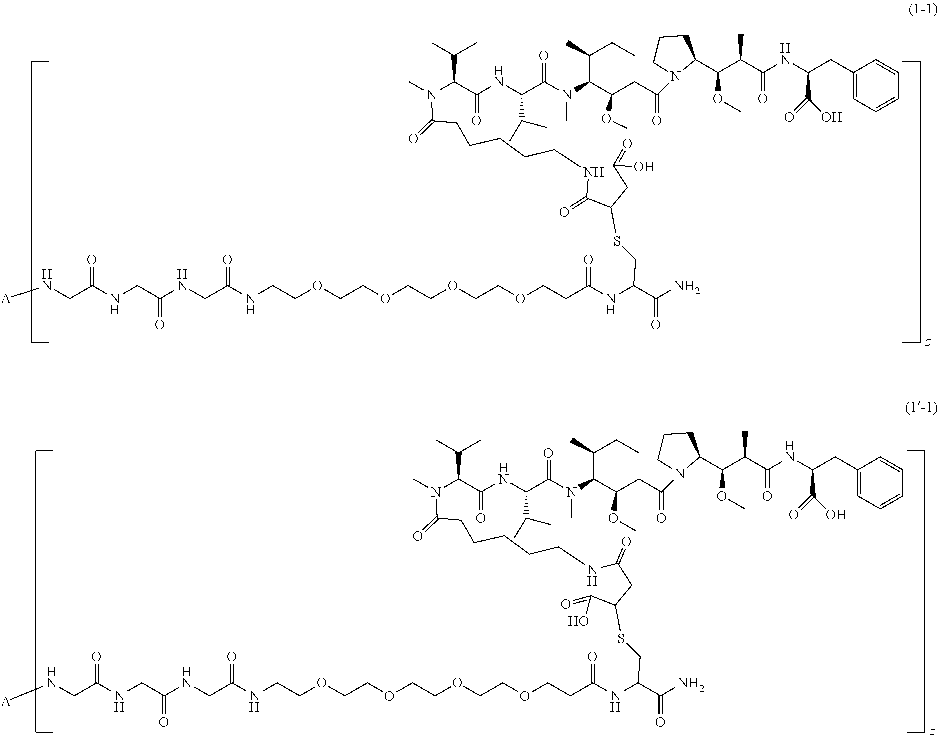
wherein z is an integer of 1 to 20;
wherein A is selected from the antibody according to any one of claims 1-12 or antigen-binding fragment thereof; preferably, A is selected from Ab0052, Ab0053, Ab0054, Ab0061, Ab0062, Ab0063 and Ab0064;
preferably, z is 2.

23. The ADC according to claim 21, wherein the ADC has the structure of Formula (2-1) or Formula (2′-1) or is a mixture thereof: 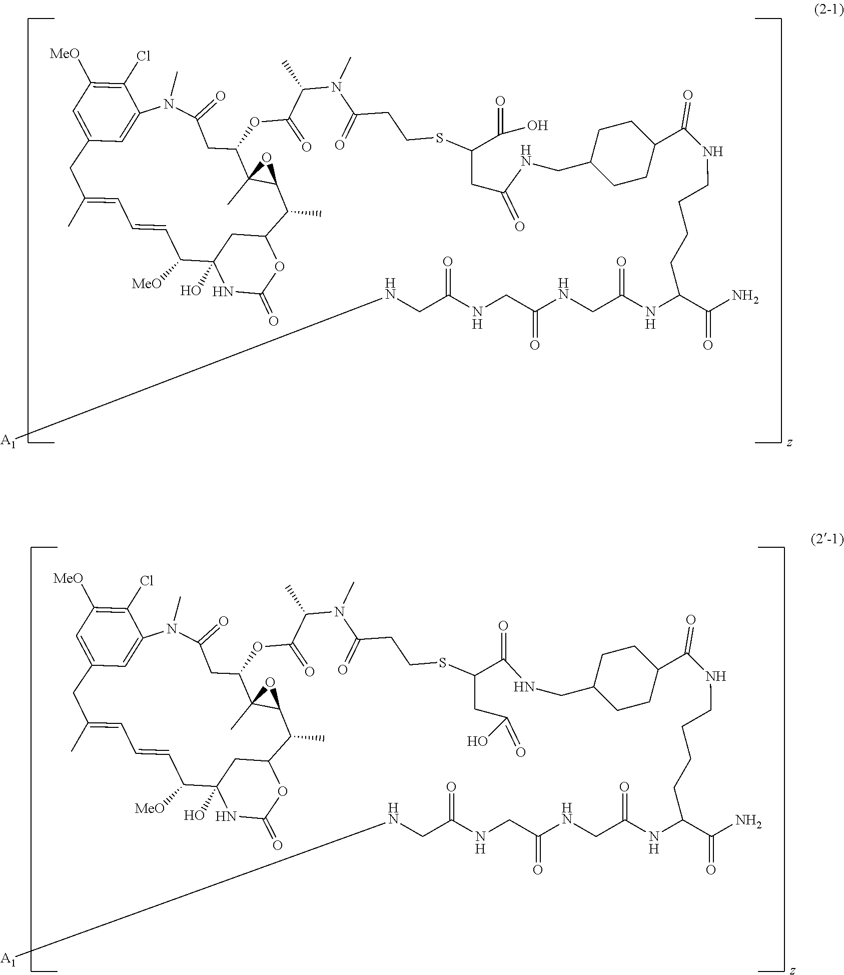
wherein z is an integer of 1 to 20;
wherein A₁ is selected from the antibody according to any one of claims 1-12 or antigen-binding fragment thereof; preferably, A₁ is selected from Ab0052, Ab0053, Ab0054, Ab0061, Ab0062, Ab0063 and Ab0064;
preferably, z is 2.

24. A chimeric antigen receptor comprising:
(i) an extracellular domain comprising the antibody according to claim 1 or antigen-binding fragment thereof,
(ii) a transmembrane domain, and
(iii) one or more intracellular stimulatory domains.

25. A host cell expressing the chimeric receptor according to claim 24, preferably, the host cell is an immune cell, more preferably T cell.

26. A pharmaceutical composition comprising:
(i) the antibody according to claim 1 or antigen-binding fragment thereof; and (ii) a pharmaceutically acceptable excipient.

27. A method of treating a disease in a subject in need thereof, comprising administering to the subject a therapeutically effective amount of the antibody according to claim 1 or antigen-binding fragment thereof, wherein the disease is TROP2-positive cancer; preferably, the TROP2-positive cancer is gastric cancer, breast cancer, urothelial cancer, lung cancer, liver cancer, endometrial cancer, head and neck cancer, or ovarian cancer, optionally wherein the method comprises administering an antibody-drug conjugate selected from DG1002 and DG1004.

28. (canceled)

29. An antibody-drug conjugate (ADC), comprising an antibody and a therapeutic agent conjugated through a linker, wherein the linker is LU104 or LU104′, which has the following structure:

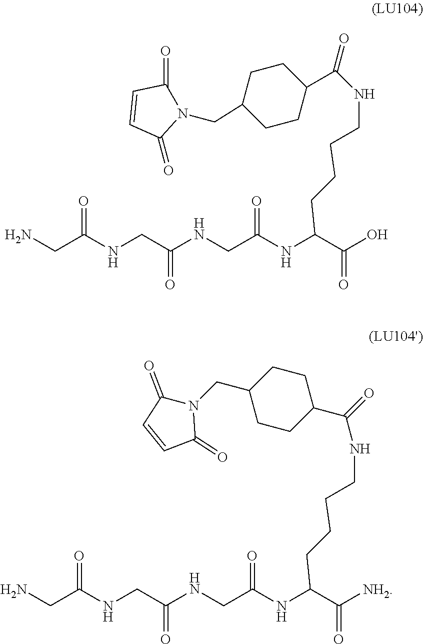 [SMILES: NCC(=O)NCC(=O)NCC(=O)NC(CCCCNC(=O)C1CCC(CN2C(=O)C=CC2=O)CC1)C(=O)O ; NCC(=O)NCC(=O)NCC(=O)NC(CCCCNC(=O)C1CCC(CN2C(=O)C=CC2=O)CC1)C(N)=O]

30. The ADC according to claim 29, wherein the ADC has the structure of Formula (II-2-1) or Formula (II-2-2) or is a mixture thereof: 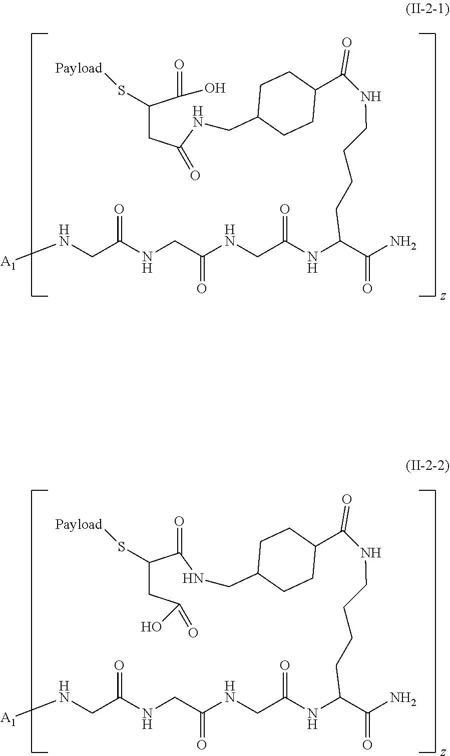
wherein z is an integer of 1 to 20;
wherein Payload is a therapeutic agent;
wherein A₁ is an antibody;
preferably, z is 2.

31. The ADC according to claim 29, wherein the therapeutic agent is a cytotoxin, preferably DM1 (mertansine).

32. The ADC according to claim 29, wherein the antibody is an antibody to TROP2; preferably, the antibody is selected from Ab0052, Ab0053, Ab0054, Ab0061, Ab0062, Ab0063, Ab0064 and Ab0002.

33. The ADC according to claim 32, wherein the ADC has the structure of Formula (2-1) or Formula (2′-1) or is a mixture thereof: 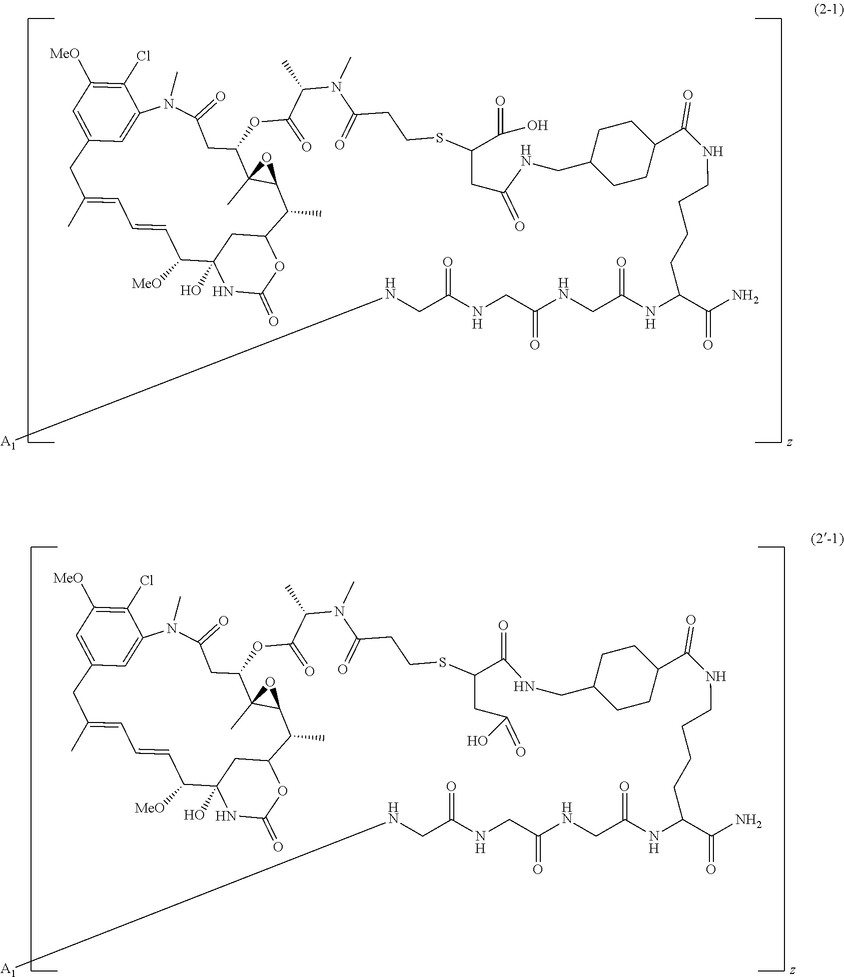
wherein z is an integer of 1 to 20;
wherein A₁ is selected from Ab0052, Ab0053, Ab0054, Ab0061, Ab0062, Ab0063, Ab0064 and Ab0002;
preferably, z is 2.

34. A method of making the antibody-drug conjugate (ADC) of claim 29, comprising
conjugating the linker to the therapeutic agent, wherein the linker comprises a maleimido moiety and the therapeutic agent has a thiol moiety, via reaction of the thiol moiety with the maleimido moiety,
reacting the intermediate thus formed with base to open the maleimide ring,
coupling this open-ring intermediate to an antibody using sortase-catalyzed coupling, and
recovering the ADC thus formed,
optionally wherein the therapeutic agent is DM1 (mertansine).

35. (canceled)

36. A method of treating a disease in a subject in need thereof, comprising administering to the subject a therapeutically effective amount of the ADC according to claim 29, wherein the disease is TROP2-positive cancer, preferably the TROP2-positive cancer is gastric cancer, breast cancer, urothelial cancer, lung cancer, liver cancer, endometrial cancer, head and neck cancer, or ovarian cancer, optionally wherein comprising administering antibody-drug conjugate DG202.

37. (canceled)
